(19) United States
(12) Patent Application Publication
(10) Pub. No.: US 2026/0158133 A1
(43) Pub. Date: Jun. 11, 2026

(54) STABILIZATION OF HUMAN IMMUNODEFICIENCY VIRUS (HIV) ENVELOPES

(71) Applicant: Duke University, Durham, NC (US)

(72) Inventors: Daniel Wrapp, Durham, NC (US); Kevin Saunders, Durham, NC (US); Barton F. Haynes, Durham, NC (US)

(21) Appl. No.: 19/123,733

(22) PCT Filed: Oct. 24, 2023
(86) PCT No.: PCT/US2023/077611

§ 371 Date: Apr. 23, 2025

Related U.S. Application Data
(60) Provisional application No. 63/418,720, filed on Oct. 24, 2022.
(60) Provisional application No. 63/434,373, filed on Dec. 21, 2022.

(51) Int. Cl. A61K 39/21; A61K 38/00; C07K 14/005; C12N 7/00

(52) U.S. Cl. CPC A61K 39/21; C07K 14/005; C12N 7/00; A61K 38/00; C07K 2319/00; C12N 2740/15022; C12N 2740/15034; C12N 2740/16034; C12N 2740/16122

(57) ABSTRACT

Disclosed are stabilized type I fusion proteins (e.g., HIV envelope proteins). For HIV-1, the stabilization of Env due to the disclosed modifications are compatible and, in some embodiments, synergistic with stabilization mutations known as SOS, IP and SOSIP. The disclosed stabilized Env proteins retain the antigenicity of the unmodified protein.

This application claims priority to U.S. Provisional Application No. 63/418,720, filed on Oct. 24, 2022, and U.S. Provisional Application No. 63/434,373, filed on Dec. 21, 2023, the entire contents of which are incorporated herein by reference.

### GOVERNMENT INTERESTS

This invention was made with government support from the Center for HIV/AIDS Vaccine Development (CHAVID) grant 5UM1AI44371.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to compositions and methods for stabilizing and/or increasing yield of lentiviral (e.g., HIV) envelope proteins.

### SEQUENCE LISTING

This application contains a Sequence Listing which has been submitted electronically in XML format. The Sequence Listing XML is incorporated herein by reference. Said XML file, created on Oct. 24, 2023, is named 2933311-000052-WO1_SL.xml and is 30,683 bytes in size.

### BACKGROUND OF THE INVENTION

The Env glycoprotein is a focus of HIV-1 vaccine development due to the role that it plays during the process of viral entry. Despite significant advances in protein engineering, many Env proteins remain recalcitrant to recombinant expression due to their inherent metastability, and propensity to change conformation from a prefusion form to a postfusion form, making biochemical and immunologic experiments difficult.

### SUMMARY OF THE INVENTION

Here we describe amino acid substitutions (e.g., proline substitutions) that significantly increase the yield of recombinantly expressed prefusion Env, including Env ectodomains, from lentiviruses. In some embodiments, substitutions referred to as “2P” increase yield of Env and can function synergistically with previously reported SOSIP mutations. The modified Env proteins do not alter the antigenicity or the overall structure of the prefusion Env trimer. While not wishing to be held to a mechanism, these mutations can increase yield through enhanced durability of the prefusion conformation of Env, rather than through boosting expression levels. We show that the 2P mutations can effectively be applied to a broad range of antigenically and evolutionarily diverse Envs. By facilitating the expression and purification of Env, the 2P mutations can be a useful tool for researchers investigating and developing medical countermeasures against viruses like HIV-1. These findings provide a useful Env stabilization platform to facilitate biochemical research and can improve the development of future HIV-1 vaccine candidates.

Disclosed is a modified type I fusion protein from a retrovirus having substituted amino acids at one or more of positions 536, 545, 568 and 569 (HBX2 numbering). The retrovirus can be a lentivirus. The retrovirus can be HIV. The retrovirus can be HIV-1, HIV-2 or SIV. The type I fusion protein from these viruses can be an Env protein, including gp160, gp120 or gp41. The Env protein can include an ectodomain.

The substituted amino acids can be at one, two, three or all of positions 536, 545, 568 and 569 in the proteins. The substituted amino acids can be at amino acid positions 536 and 545, 536 and 568, 536 and 569, 545 and 568, 545 and 569 or 568 and 569. In some embodiments, threonines at positions 568 and 569 are substituted. The amino acids at these positions can be substituted with proline. These substituted Env proteins can contain additional/other mutations elsewhere in the molecules.

The disclosed molecules can be expressed at higher levels in recombinant expression systems compared to proteins that do not have the amino acid substitutions. The disclosed molecules have a higher probability of remaining in a prefusion conformation and a lower probability of forming a postfusion conformation compared to molecules that do not have the amino acid substitutions. The disclosed molecules retain the antigenicity of the unsubstituted molecules. The disclosed molecules retain the antibody binding profile, with a panel of multiple antibodies, of the unsubstituted molecules. Coordinates from molecular models obtained using cryo-EM images were used to calculate root-mean-square-distance (RMSD) of the substituted molecules versus the unsubstituted molecules. In some embodiments, RMSD from these calculations was less than 1 Å.

Methods for constructing the disclosed proteins or modifying an existing type I fusion protein to obtain the disclosed proteins are described. Also disclosed are vaccine compostions containing the disclosed proteins, nucleic acids encoding the disclosed proteins, vectors containing the nucleic acids, and cells containing the vectors.

### BRIEF DESCRIPTION OF THE DRAWINGS

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1. Illustrates design and evaluation of proline-stabilization mutations in gp41. (A, left) A single monomer of gp41 in the prefusion conformation (PDB ID: 6VZI) is shown as ribbons, colored white. Residues that were targeted for proline stabilization are shown as colored sticks surrounded by a transparent molecular surface. (A, right) A single monomer of gp41 in the postfusion conformation (homology model based on PDB ID: 7AEJ) is shown, colored according to the prefusion monomer. (B) Size-exclusion chromatograms from a Superose 6 Increase column for each individual mutant after affinity chromatography are overlaid. (C) Size-exclusion chromatograms from a Superose 6 Increase column are shown for individual proline mutants, colored either blue or red. The combination of the two mutations is colored purple and the unmutated CH848 10.17DT DS-SOSIP is colored black and labeled “WT”.

FIG. 2. Illustrates that 2P mutations do not alter the antigenic landscape of CH848 10.17DT DS-SOSIP. ELISA binding curves for Env-directed monoclonal antibodies against CH848 10.17DT DS-SOSIP and CH848 10.17DT DS-SOSIP-2P are shown. CH848 10.17DT DS-SOSIP is shown in black (filled squares) and CH848 10.17DT DS-SOSIP-2P is shown in purple (filled circles). Data points represent the average of three replicates and the standard deviations are plotted as error bars. The y-axes show absorbance measured at 450 nm. Neutralizing antibody curves are shown in (A) and non-neutralizing antibody curves are shown in (B).

FIG. 3. Illustrates the cryo-EM structure of CH848 10.17DT DS-SOSIP-2P. (A) 2D class averages of the CH848 10.17DT DS-SOSIP-2P Env ectodomain, calculated in cryoSPARC v3, are shown. (B) The 3.73 Å-resolution reconstruction is shown from side (left) and top (right) views. Two protomers are displayed as cryo-EM map in either white or dark gray and the third protomer is shown as a ribbon diagram of the corresponding model, with gp120 colored red and gp41 colored yellow. (C) A single monomer of gp41 from CH848 10.17DT DS-SOSIP-2P is shown in yellow, aligned to a monomer of gp41 from a previously determined cryo-EM structure of CH848 10.17DT DS-SOSIP (PDB ID: 6UM7), colored purple.

FIG. 4. Illustrates investigations of the underlying mechanism of enhanced protein yield upon proline-stabilization. (A) Four cell populations were stained with a panel of Env-directed monoclonal antibodies. The average of three independent experiments is plotted±standard deviation. Each antibody has been assigned a color and is labeled above the corresponding bars. “Mock”=untransfected, “WT”=CH848 10.17DT gp160, “2P”=CH848 10.17DT 2P gp160, “2P 1559P”=CH848 10.17DT 2P 1559P gp160. (B, top left) CH848 10.17DT DS-SOSIP Tm is plotted for each round of freeze/thaw. (B, top right) DSF melting curves used to calculate CH848 10.17DT DS-SOSIP Tm are plotted, colored according to the bar graph on the left. The dotted line at 73.6° C. represents the average Tm of rounds 1˜4 and the dotted line at 66.4° C. represents the average Tm for rounds 5-10. (B, bottom left) CH848 10.17DT DS-SOSIP-2P is plotted for each round of freeze/thaw. (B, bottom right) MST melting curves used to calculate CH848 10.17DT DS-SOSIP-2P Tm are plotted, colored according to the bar graph on the left. The dotted line at 71.0° C. represents the average Tm of rounds 1-8 and the dotted line at 67.5° C. represents the average Tm for rounds 9-10. (C, top) Size-exclusion chromatograms for three aliquots of CH848 10.17DT DS-SOSIP, incubated at 42° C. for 0, 48 or 96 hours, are overlaid. The 0 hour sample is colored blue, the 48 hour sample is colored black and the 96 hour sample is colored red. Data are plotted as a percentage of the maximum mAU value from the 0 hour sample. (C, bottom) Size-exclusion chromatograms for three aliquots of CH848 10.17DT DS-SOSIP-2P, incubated at 42° C. for 0, 48 or 96 hours, are overlaid. The 0 hour sample is colored blue, the 48 hour sample is colored black and the 96 hour sample is colored red. Data are plotted as a percentage of the maximum mAU value from the 0 hour sample.

FIG. 5. Illustrates applying 2P mutations to evolutionarily diverse Envs. Size-exclusion chromatograms from a Superose 6 Increase column are shown for each construct after PGT145 affinity chromatography. Curves for the 2P-stabilized construct are colored and the curves for the corresponding non-2P-stabilized constructs (“WT”) are overlaid in black. Chromatograms were generated using a Superose 6 Increase column.

FIG. 6. Illustrates evaluation of the effects of quadruple proline-substitution on Env homogeneity and yield. Size-exclusion chromatograms from a Superose 6 Increase column are shown. CH848 10.17DT DS-SOSIP is colored black (labeled “WT”), CH848 10.17DT DS-SOSIP L568P+T569P is colored purple and CH848 10.17DT DS-SOSIP T536P+L545P+L568P+T569P is colored orange. The curves from CH848 10.17DT DS-SOSIP and CH848 10.17DT DS-SOSIP L568P+T569P are the same as those shown in FIG. 1C.

FIG. 7. Illustrates structural comparison of CoV S-2P mutations and Env 2P mutations. (A) A monomer of the gp41 subunit in the prefusion conformation (PDB ID: 6VZI) is shown as a ribbon diagram, with the HR1 colored yellow, the CH colored orange, and residues 568-569 shown as purple spheres. (B) A monomer of the gp41 subunit in the occluded-open state (PDB ID: 6CM3) is shown as a ribbon diagram, colored according to panel A.

FIG. 8. Illustrates that the 1559P mutation and the 2P mutations act synergistically to boost CH848 10.17DT Env yield. (A) Size-exclusion chromatograms from a Superose 6 Increase column are shown. CH848 10.17DT DS-SOSIP is colored red (“SOSIP”), CH848 10.17DT DS A501C+T605C+2P is colored blue (“SOS 2P”) and CH848 10.17DT DS-SOSIP-2P is colored purple (“SOSIP 2P”). The dashed box denotes the zoomed-in view that is shown in panel B.

FIG. 9. Illustrates cryo-EM data processing workflow.

FIG. 10. Illustrates cryo-EM validation. (A) FSC curves for different masking strategies employed by cryoSPARC v3 are plotted. The dotted line represents an FSC value of 0.143. (B) The viewing direction distribution plot, generated in cryoSPARC v3, is shown for the CH848 10.17DT DS-SOSIP-2P reconstruction. (C) Side (left) and top (right) views of the 3.73 Å CH848 10.17DT DS-SOSIP-2P reconstruction are shown, colored according to local resolution. (D) The cryo-EM map is shown as a transparent surface with the corresponding atomic model shown as a ribbon diagram, colored by protomer. (E) Portions of the cryo-EM map are shown as a transparent surface with the atomic model shown as a purple ribbon diagram. Side chains are shown as sticks with oxygen atoms colored red and nitrogen atoms colored blue.

FIG. 11. Illustrates flow cytometric analysis of proline-stabilized CH848 10.17DT gp160s. (A) Transiently transfected HEK293-F cells were gated to exclude doublets, debris and dead cells. mAb binding was detected using a PE-labeled goat anti-human IgG Fc secondary antibody. (B) Raw histograms for each mAb that is displayed in FIG. 4A are shown. CH848 10.17DT gp160 curves are colored orange, CH848 10.17DT 2P gp160 curves are colored purple, CH848 10.17DT 2P 1559P gp160 curves are colored blue and untransfected curves are colored gray. The number of cells, normalized to mode, is plotted on the y-axis and PE signal is plotted on the x-axis.

FIG. 12. Conservation of residues Leu568 and Thr569. A WebLogo plot, calculated using 6,599 Env sequences curated in the LANL HIV database, is shown for positions 558-579. Residues have been colored according to their chemical properties (polar=green, neutral=purple, basic=blue, acidic=red, hydrophobic=black).

FIG. 13. Representative negative-stain EM 2D class averages of CH848 10.17DT DS-SOSIP L568P+T569P, calculated in Relion 3.0, are shown.

### DETAILED DESCRIPTION OF THE INVENTION

Disclosed here are amino acid substitutions (e.g., proline substitutions) that increase the yield of recombinantly expressed prefusion Env, including Env ectodomains. By targeting the region in gp41 that connects the central helix to the rest of heptad repeat 1 (HR1), these amino acid substitutions disfavor formation of elongated alpha helices that drive transition of Env proteins to the postfusion conformation. We showed that this strategy did not alter the antigenicity or structure of prefusion Env. While not wanting to be bound to a mechanism, we showed that the substitutions can increase the yield of Env trimers by enhancing the durability of the prefusion conformation, rather than by increasing overall expression. During the harsh, prolonged conditions of transient transfection, Env ectodomains that can retain the prefusion conformation are less likely to spontaneously and irreversibly trigger to the postfusion conformation and be lost as insoluble aggregates (28). In addition to facilitating the recombinant expression and purification of recombinant prefusion Envs for use as biochemical reagents in the laboratory, the enhanced durability of example 2P Envs can have implications for future vaccine development efforts. For example, by enhancing durability of the prefusion conformation of Env, the residence of Env immunogens in the body can be increased. Improved stability of the modified Envs can stabilize vaccines prior to administration. For example, vaccines containing these Env proteins can be more stable under circumstances where it is not possible to maintain a reliable cold chain.

Many proposed vaccination regimens currently rely on extensive priming and boosting schedules, which can complicate vaccine adherence among the general population (32, 33). One reason for this fastidiousness is the difficulty associated with eliciting even autologous neutralizing antibodies (36, 37). By enhancing the durability of the prefusion conformation of Env, it can increase the residence time of Env immunogens in the body, mimicking the conditions of natural infection where natively expressed Env is perpetually present and exposed to the adaptive immune system (20). Furthermore, the results of our thermostability and forced degradation assays indicate that 2P-stabilized Envs do not need the same fastidious storage conditions that are a requirement for unstabilized Env immunogens. In addition to the advantages that this poses in a laboratory environment, it is also a consideration when thinking about administering vaccines in regions that may lack sufficient infrastructure to reliably maintain a cold chain.

We also show that the 2P stabilization strategy is not solely effective in the context of the CH848 10.17DT Env. By introducing these mutations into a diverse panel of different Env ectodomains, we were able to enhance the yield of multiple prefusion trimers, albeit to differing degrees of success depending on the variant in question. The 2P stabilization strategy can be a broadly applicable means to enhance the yield of antigenically diverse prefusion trimers and will prove to be useful for immunogen design.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

### Type I Fusion Proteins, Prefusion and Postfusion Forms, and Protein Instability

Herein are disclosed modified type I fusion proteins. In some embodiments the modified proteins have amino acid substitutions as compared to the naturally-occurring proteins. In some embodiments, the type I fusion proteins can be from viruses like influenza, some retroviruses (e.g., lentiviruses like human immunodeficiency viruses, including HIV-1 or HIV-2, and SIV), coronaviruses, Ebola viruses and the like. In some embodiments, the type I fusion proteins can be syncytins, which are proteins that can be used in mammalian cell fusions. In some embodiments, the type I fusion proteins can be made of three subunits (i.e., trimeric). The type I fusion proteins can be from HIV-1 Groups M, N or O. The HIV-1 can be from Group M and from subtype or clade A, B, C, D, F, G, H, J or K. In some embodiments, the proteins can be viral envelope proteins (Env). The Env protein can be a gp160, a gp120, or a gp41 protein. The Env protein can be an ectodomain.

HIV-1 is a lentivirus that makes use of a class I viral fusion protein, called Env, to gain entry into host cells to begin the process of integration and replication (7). Like other class I viral fusion proteins, Env is proteolytically processed into two distinct subunits, gp120 and gp41 (8). These subunits remain associated and oligomerize with other protomers to form the functional prefusion conformation of Env, which is composed of a trimer of gp120/gp41 heterodimers. The N-terminal gp120 subunit is responsible for mediating binding to both CD4 and CCR5 (9-11), while the gp41 subunit contains the hydrophobic fusion peptide and the helical heptad repeats that drive membrane fusion (12).

Due to the role that Env plays during the process of infection, it is the main target for vaccine development. However, several characteristics of Env make it a complex and problematic immunogen. To begin, it is covered by a dense glycan shield composed of N-linked glycans that hamper the elicitation of neutralizing antibodies (13). Furthermore, constant immune pressure (14) and the infidelity of the HIV-1 reverse transcriptase has resulted in sequence diversity among Env variants (15). Env has also evolved to be metastable in the prefusion conformation, such that it is primed to rapidly and irreversibly transition to the postfusion conformation (16). This inherent metastability makes it difficult to recombinantly express the Env ectodomain in the antigenically desirable prefusion conformation.

In an effort to stabilize the prefusion conformation of the Env ectodomain without altering its antigenicity, Sanders et al. developed the SOSIP mutations, composed of an engineered disulfide bond (SOS) linking the gp120 and gp41 subunits and an I559P (IP) substitution that disfavors formation of elongated postfusion helices that make up the six-helix bundle (17, 18; All amino acid numbering herein uses HXB2 numbering; Korber, Bette, et al. “Numbering positions in HIV relative to HXB2CG.” Human retroviruses and AIDS 3 (1998): 102-111). Since their initial description, the SOSIP mutations have undergone iterative improvements to enhance the thermostability and the antigenic characteristics of the prefusion trimer (19-21). Alternative and complimentary protein engineering approaches have also been described as a means of yielding improved Env immunogens (22-26). However, despite these advancements in immunogen engineering, many Env variants remain recalcitrant to recombinant in vitro expression.

In some embodiments, the modified type I fusion proteins disclosed herein have substitutions of certain amino acids that are found in the naturally-occurring or wild-type forms of the proteins. In some embodiments, the amino acid positions that are substituted can be positions in a protein that are involved in a prefusion conformation of the protein changing to a postfusion conformation. Generally, modification of the protein by substitution of amino acids at these locations disfavors conformational change of the modified protein from a prefusion to postfusion state as compared to the unmodified protein (e.g., disfavors α-helices). In some embodiments, the amino acid positions that are substituted can be found in regions of the protein that are parts of flexible loops.

In some embodiments, the amino acid positions in a retroviral (e.g., HIV-1) envelope (Env) protein can be 536, 545, 568, 569, and combinations thereof (HXB2 numbering). In various embodiments, the amino acid substitution can be made at one of positions 536, 545, 568, 569, at any two of positions 536, 545, 568, 569, at any three of positions 536, 545, 568, 569, or at all of positions 536, 545, 568, 569. In some embodiments, the amino acid substitutions can be made at one or more of positions 536, 545, 568, 569 and are the only substituted amino acids in the protein, or the only substituted amino acids in the protein that affect protein stability. In some embodiments, the amino acid positions in a retroviral (e.g., HIV-1) Env protein can be made at positions 536 and 545, 536 and 568, 536 and 569, 545 and 568, 545 and 569, or 568 and 569. In some embodiments, the substitution at position 568 can replace a leucine in the unmodified protein. In some embodiments, the substitution at position 569 can replace a threonine. In some embodiments, the leucine at position 568 and the threonine at position 569 can be replaced or substituted.

The amino acid substitutions can substitute an amino acid in a type I fusion protein for an amino acid that has a lower configurational entropy than the existing amino acid at a location. In some embodiments, the amino acids at these locations are substituted with proline. Proline substitution can reduce the conformational/configurational entropy of the protein. The cyclized side chain of proline also disfavors formation of alpha helices in proteins. In some embodiments, proline can be used as the substituting amino acid. In some embodiments, glycine can be used as the substituting amino acid.

In some embodiments, in addition to the substitutions at the 536, 545, 568 and/or 569 positions as above, the protein can have additional mutations as compared to the naturally-occurring protein. For example, the protein can have an amino acid substitution at position 559. In some embodiments, the amino acid substitution can be I559P (IP). In some embodiments, the protein can have a modification that is a disulfide bond linking a gp120 and gp41 subunit (SOS). In some embodiments, the protein can have an IP and an SOS modification (SOSIP). In some embodiments, the protein can have one or more of modifications DS (1201C+A433C), DT (N133D+N138T), chimeric BG 505 gp41, Q171K, SOSIPV6, F14 (V68I+A204V+V208L+V255L), and combinations thereof.

In some embodiments, the modified lentiviral fusion proteins can be as below. The substituted 2P amino acids are indicated by underlining/highlighting.

CH848.3.d949.10.17DTchim DS-SOSIP-2P
(SEQ ID NO: 1)
MPMGSLQPLATLYLLGMLVASVLAAENLWVTVYYGVPVWKEAKTTLFCAS
DARAYEKEVHNVWATHACVPTDPSPQELVLGNVTENFNMWKNDMVDQMHEDIISL
WDQSLKPCVKLTPLCVTLICSDATVKTGTVEEMKNCSFNTTTEIRDKEKKEYALFYK
PDIVPLSETNNTSEYRLINCNTSACTQACPKVTFEPIPIHYCAPAGYAILKCNDETFNG
TGPCSNVSTVQCTHGIRPVVSTQLLLNGSLAEKEIVIRSENLTNNAKIIIVHLHTPVEIV
CTRPNNNTRKSVRIGPGQTFYATGDIIGDIKQAHCNISEEKWNDTLQKVGIELQKHFP
NKTIKYNQSAGGDMEITTHSFNCGGEFFYCNTSNLFNGTYNGTYISTNSSANSTSTITL
QCRIKQIINMWQGVGRCMYAPPIAGNITCRSNITGLLLTRDGGTNSNETETFRPAGGD
MRDNWRSELYKYKVVKIEPLGVAPTRCKRRVVGRRRRRRAVGIGAVFLGFLGAAG
STMGAASMTLTVQARNLLSGIVQQQSNLLRAPEAQQHLLKPPVWGIKQLQARVLAV
ERYLRDQQLLGIWGCSGKLICCTNVPWNSSWSNRNLSEIWDNMTWLQWDKEISNYT
QIIYGLLEESQNQQEKNEQDLLALD
SIVcpzCAM13 Q171K DS-SOSIP-2P
(SEQ ID NO: 2)
MPMGSLQPLATLYLLGMLVASVLASDEWVTVYYGVPVWREVNTVLFCASD
AKAHSTEAHNIWATHACVPTDPNPQEVVLHNVTEDFNMWNNQMVEQMQEDISSL
WDQSLKPCVKLTPLCVTMKCSNVTRQNSTSNSNNNPTLKNNTSGKNETGVLQMKN
CTFNTTTELRDKKKKVYSLFYVDDLQSLGSGTGDTYTMINCNTTACTQACPKVSFEP
IPIHYCAPAGFAILKCNDVNFSGKGKCRNVSTVHCTHGIKPVVTTQLIINGSLATENIT
VRVNNASKNTHDWIVQLSTAVNLTCKRVGNNTRGKVQIGPGMTFYNMDHIFGDTR
KAFCELNGTTWNETLQKVRESLIKEIKANANGTYNITFEPSSGGDPEIANHMFNCGGE
FFYCDTRKMFNESEPFHENMTIPCRIRQIVNSWMRVGRCIYAPPIPGHITCNSLITGLIL
TRDHVNNTNNTFRPIGGDMKNIWRSELYKYKVVRIEPLSVAPTKCKRHTVGERRRR
RRAAFGLGALFLGFLGAAGSTMGAAAVTLTVQARQLLSGIVQQQNNLLRAPEAQQH
LLQPPVWGVKQLQARLLAVERYLQDQQILGLWGCSGKSICCTTVPWNKTWSGKSM
SDIWNNLTWQQWDKLITNYTGTIFGLLEEAQSQQEKNEKDLLELD
B41 SOSIP-2P
(SEQ ID NO: 3)
MPMGSLQPLATLYLLGMLVASVLAAAKKWVTVYYGVPVWKEATTTLFCAS
DAKAYDTEVHNVWATHACVPTDPNPQEIVLGNVTENFNMWKNNMVEQMHEDIISL
WDQSLKPCVKLTPLCVTLNCNNVNTNNTNNSTNATISDWEKMETGEMKNCSFNVT
TSIRDKIKKEYALFYKLDVVPLENKNNINNTNITNYRLINCNTSVITQACPKVSFEPIPI
HYCAPAGFAILKCNSKTFNGSGPCTNVSTVQCTHGIRPVVSTQLLLNGSLAEEEIVIRS
ENITDNAKTIIVQLNEAVEINCTRPNNNTRKSIHIGPGRAFYATGDIIGNIRQAHCNISK
ARWNETLGQIVAKLEEQFPNKTIIFNHSSGGDPEIVTHSFNCGGEFFYCNTTPLFNSTW
NNTRTDDYPTGGEQNITLQCRIKQIINMWQGVGKAMYAPPIRGQIRCSSNITGLLLTR
DGGRDQNGTETFRPGGGNMRDNWRSELYKYKVVKIEPLGIAPTACKRRVVQRRRR
RRAVGLGAFILGFLGAAGSTMGAASMALTVQARLLLSGIVQQQNNLLRAPEAQQHM
LQPPVWGIKQLQARVLAVERYLRDQQLLGIWGCSGKIICCTNVPWNDSWSNKTINEI
WDNMTWMQWEKEIDNYTQHIYTLLEVSQIQQEKNEQELLELD
JRFL SOSIPv6-2P
(SEQ ID NO: 4)
MPMGSLQPLATLYLLGMLVASCLGVEKLWVTVYYGVPVWKEACTTLFCAS
DAKAYDTKVRNVWATHCCVPTDPNPQEVVLENVTEHFNMWKNNMVEQMQEDIIS
LWDQSLKPCVKLTPLCVTLNCKDVNATNTTNDSEGTMERGEIKNCSFNITTSIRDKV
QKEYALFYKLDVVPIDNNNTSYRLISCDTSVITQACPKISFEPIPIHYCAPAGFAILKCN
DKTFNGKGPCKNVSTVQCTHGIRPVVSTQLLLNGSLAEEEVVIRSDNFTNNAKTIIVQ
LKESVEINCTRPNNNTRKSIHIGPGRWFYTTGEIIGDIRQAHCNISRAKWNDTLKQIVI
KLREQFENKTIVFNHSSGGDPEIVMHSFNCGGEFFYCNSTQLFNSTWNNNTEGSNNT
EGNTITLPCRIKQIINMWQEIGKAMYAPPIRGQIRCSSNITGLLLTRDGGINENGTEIFR
PGGGDMRDNWRSELYKYKVVKIEPLGVAPTKCKRRVVGRRRRRRAVGIGAVFLGF
LGAAGSTMGAASMTLTVQARQLLSGIVQQQNNCLRAPECQQRMLQPPVWGIKQLQ
ARVLAVERYLGDQQLLGIWGCSGKLICCTAVPWNASWSNKSLDRIWNNMTWMEW
EREIDNYTSEIYTLIEESQNQQEKNEQELLELD
T250-4 DS-SOSIP-2P
(SEQ ID NO: 5)
MDAMKRGLCCVLLLCGAVFVSPSQEIHARFRRGARAEKLWVTVYYGVPVW
READTTLFCASDAKGYDTEAHNVWATHACVPTDPRPQEMYLENVTENFNMWKNS
MVEQMHTDIISLWDESLKPCVKLTPLCVTLDCQAFNSSSHTNSSIAMQEMKNCSFNV
TTELRDKKKKEYSFFYKTDIEQINKNGRQYRLINCNTSACTQACPKVSFEPIPIHFCAP
AGFAILKCNEKHFNGKGPCKNVSTVQCTHGIKPVVSTQLLLNGSLAEEEVVIRVENTI
DNAKTIIVQLAKPVKINCTRPNNNTRKSIRIGPGQTFYATGDIIGNIRKAYCNVSKREW
NNTLQQVAAQLSKSFNNTKIVFEKHSGGDLEVITHSFVCGGEFFYCNTSGLFNSTWT
NSTWTNSTTGSNGTESNDTITLQCEIKQFINMWQRVGRCMYAPPIPGVIRCESDITGL
LLTRDGPNSTQNETFRPGGGDMRDNWRSELYKYKVVQIEPLGVAPTHCKRRVVERR
RRRRAVGLGAVFFGFLGAAGSTMGAASITLTVQARQLLSGIVQQQSNLLKAPEAQQ
QLLRPPVWGIKQLQARVLALERYLKDQQLLGIWGCSGKLICCTTVPWNSSWSNKNY
TDIWDNMTWLQWDREISNYTDEIYRLIEQSQNQQEKNEQDLLALD
CH505w24 F14 DS-SOSIP-2P
(SEQ ID NO: 6)
MPMGSLQPLATLYLLGMLVASVLAAENLWVTVYYGVPVWKEAKTTLFCAS
DAKAYEKEVHNIWATHACVPTDPNPQEMVLKNVTENFNMWKNDMVDQMHEDVIS
LWDQSLKPCVKLTPLCVTLNCTNATANATASNSSIIEGMKNCSFNITTELRDKREKK
NALFYKLDIVQLDGNSSQYRLINCNTSVITQVCPKVSFDPIPIHYCAPAGYAILKCNNK
TFTGTGPCNNVSTVQCTHGIKPVLSTQLLLNGSLAEGEIIIRSENITNNGKTIIVQLNES
VKIECTRPNNKTRTSIRIGPGQAFYATGQVIGNIREAYCNISESKWNETLQRVSKKLKE
YFPHKNITFQPSSGGDLEITTHSFNCGGEFFYCNTSSLFNRTYMANSTDMANSTETNS
TRIITIHCRIKQIINMWQEVGRAMYAPPIAGNITCISNITGLLLTRDGGKNNTDTETFRP
GGGNMKDNWRSELYKYKVVEVKPLGVAPTNCRRRVVERRRRRRAVGMGAVFLGF
LGAAGSTMGAASITLTVQARQLLSGIVQQQSNLLKAPEAQQHMLKPPVWGIKQLQA
RVLALERYLKDQQLLGMWGCSGKLICCTNVYWNSSWSNKTYGDIWDNMTWMQW
EREISNYTEIIYELLEESQNQQEKNEQDLLALD

In some embodiments, the modified lentiviral fusion proteins can be as below (i.e., mrna3_CAP256SU_UCA_OPT_4.0, mrna4_CAP256SU_UCA_OPT_4.0 and mrna5_CAP256SU_UCA_OPT_4.0). The substituted 2P amino acids are indicated by underlining/highlighting.

>mrna3_CAP256SU_UCA_OPT_4.0
(SEQ ID NO: 7)
MAISGVPVLGFFIIAVLMSAQESWAGLWVTVYYGVPVWREAKTTLFCASDA
KSYEKEVANIWATHACVPTDPNPQELVLKNVTENFNMWKNDMVDQMHEDIISLWD
QSLKPCVKLTPLCVTLHCSTYNNTHNISKEIKNCSFNATTELRDKRRKEYALFYRLDI
VPLNKNGRQYRLINCNTSVCTQVCPKLTFDPIPIHYCAPAGYAILKCNNKTENGTGPC
NNVSTVQCTHGIKPVLSTQLLLNGSLAEEEIIIRSENLTDNVKTIIVHLNESVEINCTRP
NNNTRKSIRIGPGQTFYATGDIIGDIRQAHCNISEIKWNKTLQRVSEKLREHFNKTIIFN
QSSGGDLEITTHSFNCGGEFFYCNTSDLFFNKTFNETYSTGSNSTNSNITLPCRIKQIIN
MWQEVGRCMYASPIAGEITCKSNITGLLLTRDGGGNNSTEETFRPGGGNMRDNWRS
ELYKYKVVEVKPLGIAPTECRRRVVQGGGGSGGGGSAVVGLGAVFLGFLGTAGSTM
GAASITLTVQARQLLSGIVQQQSNLLRAPEAQQHMLQPPVWGIKQLQARVLTIERYA
KDQQLLGMWGCSGKLICCTNVYWNSSWSNKTYNEIWDNMTWMQWDREIDNYTD
TIYKLLEVSQKQQESNEKDLLALDSWNNLWNWFDISKWLWYIKIFIMIVGGLIGLRII
FAVLSLVNRVRQGIRPVFSSPPSYFQ
>mrna4_CAP256SU_UCA_OPT_4.0
(SEQ ID NO: 8)
MAISGVPVLGFFIIAVLMSAQESWAGLWVTVYYGVPVWREAKTTLFCASDA
KSYEKEVANIWATHACVPTDPNPQELVLKNVTENFNMWKNDMVDQMHEDIISLWD
QSLKPCVKLTPLCVTLHCSTYNNTHNISKEIKNCSFNATTELRDKRRKEYALFYRLDI
VPLNKNGRQYRLINCNTSVCTQVCPKLTFDPIPIHYCAPAGYAILKCNNKTFNGTGPC
NNVSTVQCTHGIKPVLSTQLLLNGSLAEEEIIIRSENLTDNVKTIIVHLNESVEINCTRP
NNMTRKSIRIGPGQTFYALGDIIGDIRQPHCNISEIKWNKTLQRVSEKLREHFNKTIIFN
QSSGGDLEITTHSFNCGGEFFYCNTSDLFFNKTFNETYSTGSNSTNSNITLPCRIKQIIN
MWQEVGRCMYAPPIAGNITCKSNITGLLLTRDGGGNNSTEETFRPGGGNMRDNWRS
ELYKYKVVEVKPLGIAPTECRRRVVQGGGGSGGGGSAVVGLGAVFLGFLGTAGSTM
GAASNTLTVQARQLLSGIVQQQSNLLRAPEAQQHMLQPPVWGFKQLQARVLTIERY
AEVQQLLGMWGCSGKLICCTNVPWNSSWSNKTYNEIWDNMTWMQWDREIGNYTD
TIYKLLEVSQFQQEINEKDLLALDSWNNLWNWFDISKWLWYIKIFIMIVGGLIGLRIIF
AVLSLVNRVRQGIRPVFSSPPSYFQ
mrna5_CAP256SU_UCA_OPT
(SEQ ID NO: 9)
MAISGVPVLGFFIIAVLMSAQESWAGLWVTVYYGVPVWREAKTTLFCASDA
KSYEKEVHNVWATHACVPTDPNPQELVLKNVTENFNMWKNDMVDQMHEDIISLW
DQSLKPCVKLTPLCVTLHCSTYNNTHNISKEIKNCSFNATTELRDKRRKEYALFYRLD
IVPLNKNGRQYRLINCNTSVCTQICPKVTFDPIPIHYCAPAGYAILKCNNKTFNGTGPC
NNVSTVQCTHGIKPVVSTQLLLNGSLAEEEIIIRSENLTDNVKTIIVHLNESVEINCTRP
NNMTRKSIRIGPGQTFYALGDIIGDIRQPHCNISEIKWNKTLQRVSEKLREHFNKTIIFN
QSSGGDLEITTHSFNCGGEFFYCNTSDLFFNKTFNETYSTGSNSTNSNITLPCRIKQIIN
MWQEVGRCMYAPPIAGNITCKSNITGLLLTRDGGGNNSTEETFRPGGGNMRDNWRS
ELYKYKVVEVKPLGIAPTECRRRVVQGGGGSGGGGSAVVGLGAVFLGFLGTAGSTM
GAASNTLTVQARQLLSGIVQQQSNLLRAPEAQQHMLQPPVWGFKQLQARVLAIERY
LEVQQLLGMWGCSGKLICCTNVPWNSSWSNKTYNEIWDNMTWMQWDREIGNYTD
TIYKLLEVSQFQQEINEKDLLALDSWNNLWNWFDISKWLWYIKIFIMIVGGLIGLRIIF
AVLSLVNRVRQGIRPVFSSPPSYFQ

The nucleic acid sequences encoding the mrna3, mrna4 and mrna5 proteins shown above are shown below (i.e., 2560_pUC-ccTEV-co.mRNA3_CAP256SU_UCA_OPT_4.0-A101, 2560_pUC-ccTEV-co.mRNA4_CAP256SU_UCA_OPT_4.0-A101 and 2560_pUC-ccTEV-co.mRNA5_CAP256SU_UCA_OPT_4.0-A101).

>2560_pUC-ccTEV-co.mRNA3_CAP256SU_UCA_OPT_4.0-A101
(SEQ ID NO: 10)
aGcATAAAAGTCTCAACACAACATATACAAAACAAACGAATCTCAAGCAA
TCAAGCATTCTACTTCTATTGCAGCAATTTAAATCATTTCTTTTAAAGCAAAAGCA
ATTTTCTGAAAATTTTCACCATTTACGAACGATAGCGCTgccaccATGGCCATCTCC
GGCGTGCCCGTGCTGGGCTTCTTCATCATCGCCGTGCTGATGTCCGCCCAGGAGT
CCTGGGCCGGCCTGTGGGTGACCGTGTACTACGGCGTGCCCGTGTGGCGCGAGG
CCAAGACCACCCTGTTCTGCGCCTCCGACGCCAAGTCCTACGAGAAGGAGGTGG
CCAACATCTGGGCCACCCACGCCTGCGTGCCCACCGACCCCAACCCCCAGGAGC
TGGTGCTGAAGAACGTGACCGAGAACTTCAACATGTGGAAGAACGACATGGTGG
ACCAGATGCACGAGGACATCATCTCCCTGTGGGACCAGTCCCTGAAGCCCTGCGT
GAAGCTGACCCCCCTGTGCGTGACCCTGCACTGCTCCACCTACAACAACACCCAC
AACATCTCCAAGGAGATCAAGAACTGCTCCTTCAACGCCACCACCGAGCTGCGC
GACAAGCGCCGCAAGGAGTACGCCCTGTTCTACCGCCTGGACATCGTGCCCCTG
AACAAGAACGGCCGCCAGTACCGCCTGATCAACTGCAACACCTCCGTGTGCACC
CAGGTGTGCCCCAAGCTGACCTTCGACCCCATCCCCATCCACTACTGCGCCCCCG
CCGGCTACGCCATCCTGAAGTGCAACAACAAGACCTTCAACGGCACCGGCCCCT
GCAACAACGTGTCCACCGTGCAGTGCACCCACGGCATCAAGCCCGTGCTGTCCA
CCCAGCTGCTGCTGAACGGCTCCCTGGCCGAGGAGGAGATCATCATCCGCTCCG
AGAACCTGACCGACAACGTGAAGACCATCATCGTGCACCTGAACGAGTCCGTGG
AGATCAACTGCACCCGCCCCAACAACAACACCCGCAAGTCCATCCGCATCGGCC
CCGGCCAGACCTTCTACGCCACCGGCGACATCATCGGCGACATCCGCCAGGCCC
ACTGCAACATCTCCGAGATCAAGTGGAACAAGACCCTGCAGCGCGTGTCCGAGA
AGCTGCGCGAGCACTTCAACAAGACCATCATCTTCAACCAGTCCTCCGGCGGCG
ACCTGGAGATCACCACCCACTCCTTCAACTGCGGCGGCGAGTTCTTCTACTGCAA
CACCTCCGACCTGTTCTTCAACAAGACCTTCAACGAGACCTACTCCACCGGCTCC
AACTCCACCAACTCCAACATCACCCTGCCCTGCCGCATCAAGCAGATCATCAACA
TGTGGCAGGAGGTGGGCCGCTGCATGTACGCCTCCCCCATCGCCGGCGAGATCA
CCTGCAAGTCCAACATCACCGGCCTGCTGCTGACCCGCGACGGCGGCGGCAACA
ACTCCACCGAGGAGACCTTCCGCCCCGGCGGCGGCAACATGCGCGACAACTGGC
GCTCCGAGCTGTACAAGTACAAGGTGGTGGAGGTGAAGCCCCTGGGCATCGCCC
CCACCGAGTGCCGCCGCCGCGTGGTGCAGGGCGGCGGCGGCTCCGGCGGCGGCG
GCTCCGCCGTGGTGGGCCTGGGCGCCGTGTTCCTGGGCTTCCTGGGCACCGCCGG
CTCCACCATGGGCGCCGCCTCCATCACCCTGACCGTGCAGGCCCGCCAGCTGCTG
TCCGGCATCGTGCAGCAGCAGTCCAACCTGCTGCGCGCCCCCGAGGCCCAGCAG
CACATGCTGCAGCCCCCCGTGTGGGGCATCAAGCAGCTGCAGGCCCGCGTGCTG
ACCATCGAGCGCTACGCCAAGGACCAGCAGCTGCTGGGCATGTGGGGCTGCTCC
GGCAAGCTGATCTGCTGCACCAACGTGTACTGGAACTCCTCCTGGTCCAACAAGA
CCTACAACGAGATCTGGGACAACATGACCTGGATGCAGTGGGACCGCGAGATCG
ACAACTACACCGACACCATCTACAAGCTGCTGGAGGTGTCCCAGAAGCAGCAGG
AGTCCAACGAGAAGGACCTGCTGGCCCTGGACTCCTGGAACAACCTGTGGAACT
GGTTCGACATCTCCAAGTGGCTGTGGTACATCAAGATCTTCATCATGATCGTGGG
CGGCCTGATCGGCCTGCGCATCATCTTCGCCGTGCTGTCCCTGGTGAACCGCGTG
CGCCAGGGCATCCGCCCCGTGTTCTCCTCCCCCCCCTCCTACTTCCAGtaataaactagtA
GTGACTGACTAGGATCTGGTTACCACTAAACCAGCCTCAAGAACACCCGAATGG
AGTCTCTAAGCTACATAATACCAACTTACACTTACAAAATGTTGTCCCCCAAAAT
GTAGCCATTCGTATCTGCTCCTAATAAAAAGAAAGTTTCTTCACATTCTAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAA
>2560_pUC-ccTEV-co.mRNA4_CAP256SU_UCA_OPT_4.0-A101
(SEQ ID NO: 11)
aGcATAAAAGTCTCAACACAACATATACAAAACAAACGAATCTCAAGCAA
TCAAGCATTCTACTTCTATTGCAGCAATTTAAATCATTTCTTTTAAAGCAAAAGCA
ATTTTCTGAAAATTTTCACCATTTACGAACGATAGCGCTgccaccATGGCCATCTCC
GGCGTGCCCGTGCTGGGCTTCTTCATCATCGCCGTGCTGATGTCCGCCCAGGAGT
CCTGGGCCGGCCTGTGGGTGACCGTGTACTACGGCGTGCCCGTGTGGCGCGAGG
CCAAGACCACCCTGTTCTGCGCCTCCGACGCCAAGTCCTACGAGAAGGAGGTGG
CCAACATCTGGGCCACCCACGCCTGCGTGCCCACCGACCCCAACCCCCAGGAGC
TGGTGCTGAAGAACGTGACCGAGAACTTCAACATGTGGAAGAACGACATGGTGG
ACCAGATGCACGAGGACATCATCTCCCTGTGGGACCAGTCCCTGAAGCCCTGCGT
GAAGCTGACCCCCCTGTGCGTGACCCTGCACTGCTCCACCTACAACAACACCCAC
AACATCTCCAAGGAGATCAAGAACTGCTCCTTCAACGCCACCACCGAGCTGCGC
GACAAGCGCCGCAAGGAGTACGCCCTGTTCTACCGCCTGGACATCGTGCCCCTG
AACAAGAACGGCCGCCAGTACCGCCTGATCAACTGCAACACCTCCGTGTGCACC
CAGGTGTGCCCCAAGCTGACCTTCGACCCCATCCCCATCCACTACTGCGCCCCCG
CCGGCTACGCCATCCTGAAGTGCAACAACAAGACCTTCAACGGCACCGGCCCCT
GCAACAACGTGTCCACCGTGCAGTGCACCCACGGCATCAAGCCCGTGCTGTCCA
CCCAGCTGCTGCTGAACGGCTCCCTGGCCGAGGAGGAGATCATCATCCGCTCCG
AGAACCTGACCGACAACGTGAAGACCATCATCGTGCACCTGAACGAGTCCGTGG
AGATCAACTGCACCCGCCCCAACAACATGACCCGCAAGTCCATCCGCATCGGCC
CCGGCCAGACCTTCTACGCCCTGGGCGACATCATCGGCGACATCCGCCAGCCCCA
CTGCAACATCTCCGAGATCAAGTGGAACAAGACCCTGCAGCGCGTGTCCGAGAA
GCTGCGCGAGCACTTCAACAAGACCATCATCTTCAACCAGTCCTCCGGCGGCGAC
CTGGAGATCACCACCCACTCCTTCAACTGCGGCGGCGAGTTCTTCTACTGCAACA
CCTCCGACCTGTTCTTCAACAAGACCTTCAACGAGACCTACTCCACCGGCTCCAA
CTCCACCAACTCCAACATCACCCTGCCCTGCCGCATCAAGCAGATCATCAACATG
TGGCAGGAGGTGGGCCGCTGCATGTACGCCCCCCCCATCGCCGGCAACATCACC
TGCAAGTCCAACATCACCGGCCTGCTGCTGACCCGCGACGGCGGCGGCAACAAC
TCCACCGAGGAGACCTTCCGCCCCGGCGGCGGCAACATGCGCGACAACTGGCGC
TCCGAGCTGTACAAGTACAAGGTGGTGGAGGTGAAGCCCCTGGGCATCGCCCCC
ACCGAGTGCCGCCGCCGCGTGGTGCAGGGCGGCGGCGGCTCCGGCGGCGGCGGC
TCCGCCGTGGTGGGCCTGGGCGCCGTGTTCCTGGGCTTCCTGGGCACCGCCGGCT
CCACCATGGGCGCCGCCTCCAACACCCTGACCGTGCAGGCCCGCCAGCTGCTGTC
CGGCATCGTGCAGCAGCAGTCCAACCTGCTGCGCGCCCCCGAGGCCCAGCAGCA
CATGCTGCAGCCCCCCGTGTGGGGCTTCAAGCAGCTGCAGGCCCGCGTGCTGACC
ATCGAGCGgTACGCCGAGGTGCAGCAGCTGCTGGGCATGTGGGGCTGCTCCGGC
AAGCTGATCTGCTGCACCAACGTGCCCTGGAACTCCTCCTGGTCCAACAAGACCT
ACAACGAGATCTGGGACAACATGACCTGGATGCAGTGGGACCGCGAGATCGGCA
ACTACACCGACACCATCTACAAGCTGCTGGAGGTGTCCCAGTTCCAGCAGGAGA
TCAACGAGAAGGACCTGCTGGCCCTGGACTCCTGGAACAACCTGTGGAACTGGT
TCGACATCTCCAAGTGGCTGTGGTACATCAAGATCTTCATCATGATCGTGGGCGG
CCTGATCGGCCTGCGCATCATCTTCGCCGTGCTGTCCCTGGTGAACCGCGTGCGC
CAGGGCATCCGCCCCGTGTTCTCCTCCCCCCCCTCCTACTTCCAGtaataaactagtAGTG
ACTGACTAGGATCTGGTTACCACTAAACCAGCCTCAAGAACACCCGAATGGAGT
CTCTAAGCTACATAATACCAACTTACACTTACAAAATGTTGTCCCCCAAAATGTA
GCCATTCGTATCTGCTCCTAATAAAAAGAAAGTTTCTTCACATTCTAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAA
>2560_pUC-ccTEV-co.mRNA5_CAP256SU_UCA_OPT_4.0-A101
(SEQ ID NO: 12)
aGcATAAAAGTCTCAACACAACATATACAAAACAAACGAATCTCAAGCAA
TCAAGCATTCTACTTCTATTGCAGCAATTTAAATCATTTCTTTTAAAGCAAAAGCA
ATTTTCTGAAAATTTTCACCATTTACGAACGATAGCGCTgccaccATGGCCATCTCC
GGCGTGCCCGTGCTGGGCTTCTTCATCATCGCCGTGCTGATGTCCGCCCAGGAGT
CCTGGGCCGGCCTGTGGGTGACCGTGTACTACGGCGTGCCCGTGTGGCGCGAGG
CCAAGACCACCCTGTTCTGCGCCTCCGACGCCAAGTCCTACGAGAAGGAGGTGC
ACAACGTGTGGGCCACCCACGCCTGCGTGCCCACCGACCCCAACCCCCAGGAGC
TGGTGCTGAAGAACGTGACCGAGAACTTCAACATGTGGAAGAACGACATGGTGG
ACCAGATGCACGAGGACATCATCTCCCTGTGGGACCAGTCCCTGAAGCCCTGCGT
GAAGCTGACCCCCCTGTGCGTGACCCTGCACTGCTCCACCTACAACAACACCCAC
AACATCTCCAAGGAGATCAAGAACTGCTCCTTCAACGCCACCACCGAGCTGCGC
GACAAGCGCCGCAAGGAGTACGCCCTGTTCTACCGCCTGGACATCGTGCCCCTG
AACAAGAACGGCCGCCAGTACCGCCTGATCAACTGCAACACCTCCGTGTGCACC
CAGATCTGCCCCAAGGTGACCTTCGACCCCATCCCCATCCACTACTGCGCCCCCG
CCGGCTACGCCATCCTGAAGTGCAACAACAAGACCTTCAACGGCACCGGCCCCT
GCAACAACGTGTCCACCGTGCAGTGCACCCACGGCATCAAGCCCGTGGTGTCCA
CCCAGCTGCTGCTGAACGGCTCCCTGGCCGAGGAGGAGATCATCATCCGCTCCG
AGAACCTGACCGACAACGTGAAGACCATCATCGTGCACCTGAACGAGTCCGTGG
AGATCAACTGCACCCGCCCCAACAACATGACCCGCAAGTCCATCCGCATCGGCC
CCGGCCAGACCTTCTACGCCCTGGGCGACATCATCGGCGACATCCGCCAGCCCCA
CTGCAACATCTCCGAGATCAAGTGGAACAAGACCCTGCAGCGCGTGTCCGAGAA
GCTGCGCGAGCACTTCAACAAGACCATCATCTTCAACCAGTCCTCCGGCGGCGAC
CTGGAGATCACCACCCACTCCTTCAACTGCGGCGGCGAGTTCTTCTACTGCAACA
CCTCCGACCTGTTCTTCAACAAGACCTTCAACGAGACCTACTCCACCGGCTCCAA
CTCCACCAACTCCAACATCACCCTGCCCTGCCGCATCAAGCAGATCATCAACATG
TGGCAGGAGGTGGGCCGCTGCATGTACGCCCCCCCCATCGCCGGCAACATCACC
TGCAAGTCCAACATCACCGGCCTGCTGCTGACCCGCGACGGCGGCGGCAACAAC
TCCACCGAGGAGACCTTCCGCCCCGGCGGCGGCAACATGCGCGACAACTGGCGC
TCCGAGCTGTACAAGTACAAGGTGGTGGAGGTGAAGCCCCTGGGCATCGCCCCC
ACCGAGTGCCGCCGCCGCGTGGTGCAGGGCGGCGGCGGCTCCGGCGGCGGCGGC
TCCGCCGTGGTGGGCCTGGGCGCCGTGTTCCTGGGCTTCCTGGGCACCGCCGGCT
CCACCATGGGCGCCGCCTCCAACACCCTGACCGTGCAGGCCCGCCAGCTGCTGTC
CGGCATCGTGCAGCAGCAGTCCAACCTGCTGCGCGCCCCCGAGGCCCAGCAGCA
CATGCTGCAGCCCCCCGTGTGGGGCTTCAAGCAGCTGCAGGCCCGCGTGCTGGCC
ATCGAGCGCTACCTGGAGGTGCAGCAGCTGCTGGGCATGTGGGGCTGCTCCGGC
AAGCTGATCTGCTGCACCAACGTGCCCTGGAACTCCTCCTGGTCCAACAAGACCT
ACAACGAGATCTGGGACAACATGACCTGGATGCAGTGGGACCGCGAGATCGGCA
ACTACACCGACACCATCTACAAGCTGCTGGAGGTGTCCCAGTTCCAGCAGGAGA
TCAACGAGAAGGACCTGCTGGCCCTGGACTCCTGGAACAACCTGTGGAACTGGT
TCGACATCTCCAAGTGGCTGTGGTACATCAAGATCTTCATCATGATCGTGGGCGG
CCTGATCGGCCTGCGCATCATCTTCGCCGTGCTGTCCCTGGTGAACCGCGTGCGC
CAGGGCATCCGCCCCGTGTTCTCCTCCCCCCCCTCCTACTTCCAGtaataaactagtAGTG
ACTGACTAGGATCTGGTTACCACTAAACCAGCCTCAAGAACACCCGAATGGAGT
CTCTAAGCTACATAATACCAACTTACACTTACAAAATGTTGTCCCCCAAAATGTA
GCCATTCGTATCTGCTCCTAATAAAAAGAAAGTTTCTTCACATTCTAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAA

Table 1, below, discloses amino acid modifications made in the mrna3_CAP256SU_UCA_OPT_4.0, mrna4_CAP256SU_UCA_OPT 4.0 and mrna5_CAP256SU_UCA_OPT_4.0 amino acid sequences, shown above.

TABLE 1
Modifications in Amino Acid Sequence of Env

| Modification | Mutations/Sequence | | |
|---|---|---|---|
| name | (HXB2 numbering) | Purpose | Reference |
| Y712I | Y712I | Increase expression of Env | Labranche et al. J. |
| | | on cell surface | Virol. 69(9): |
| | | | 5217-5227 (1995) |
| Sodroski | H66A A582T | Prevent CD4-induced | Pacheco et al. J Virol. |
| | L587A | conformations | 91(5): e02219-16 |
| | | | (2017). |
| F14 | A204V V208L V68I | Prevent CD4-induced | Henderson et al. Nat |
| | V255L | conformations | Commun. 11: 520 |
| | | | (2020). |
| SOSIP | A501C T605C | Stabilize prefusion | Sanders et al. J. |
| | I559P | conformation | Virol. 76, 8875-8889 |
| | | | (2002) |
| SOS | A501C T605C | Stabilize prefusion | Sanders et al. J. |
| | | conformation | Virol. 76, 8875-8889 |
| | | | (2002) |
| DS | I201C A433C | Fix prefusion conformation | Kwon et al. Nat |
| | | | Struct Mol Biol |
| | | | 22:522-531 (2015). |
| 2P | L568P T569P | Stabliizes the prefusion | Wrapp et al. |
| | | conformation of gp41. | unpublished |
| | | Sometimes referred to as 2P | |
| 3mut | N302M T320L | Stabilize trimer apex, | Chuang, G-Y et al. J |
| | A329P | improve thermostability | Virol 94: e00074-20 |
| | | | (2020) |
| 2G | D636G T569G | Prevent postfusion gp41 | Guenaga J., et al. |
| | | helical transition | Immunity 46(5): |
| | | | 792-803.e3 (2017). |
| RnS | E442N, S437P, | Replace rare and/or | Gorman J., et al. Cell |
| | A204I, I573F, | destabilizing mutations from | Reports 31(1):107488 |
| | K588E, D589V, | wildtype Env. (Specific to | (2020). |
| | Y609P, K651F, | CAP256wk34.80 Env, but we | |
| | S655I, I535N | also used for CAP256SU | |
| | | based on its high similarity | |
| | | to the former. Due to | |
| | | conflict with F14 mutation | |
| | | A204V, when RnS are combined | |
| | | with F14, the A204V in F14 | |
| | | was used.) | |
| Signal Peptide | Replace wildtype | Improve expression of mRNA | Mohamad-Gabriel |
| #1 | signal peptide with | constructs. | Alameh, Drew |
| | MAISGVPVLGFFII | | Weissman et al. |
| | AVLMSAQESWA | | unpublished |
| | (SEQ ID NO: 13) | | |
| SIVMac CT | Replace wildtype | Improve expression of Env | Postler T.S., |
| | cytoplasmic tail | on cell-surface when | Desrosiers R.C. J. |
| | (HXB2 713-854) | expressed by mRNA | Virol. 87(1): 2-15 |
| | with truncated | immunogens. | (2013). |
| | SIVMac cytoplasmic | | |
| | tail | | |
| | RPVFSSPPSYFQ | | |
| | (SEQ ID NO: 14) | | |
| 2560_pUC- | aGcATAAAAGTCT | Optimal 5′ UTR sequence for | Mohamad-Gabriel |
| ccTEV-A101 | CAACACAACATA | mRNA stability and half-life | Alameh, Drew |
| 5′UTR | TACAAAACAAAC | from screens by Mohamad- | Weissman et al. |
| | GAATCTCAAGCA | Gabriel Alameh, Drew | unpublished |
| | ATCAAGCATTCT | Weissman et al. | |
| | ACTTCTATTGCA | | |
| | GCAATTTAAATC | | |
| | ATTTCTTTTAAA | | |
| | GCAAAAGCAATT | | |
| | TTCTGAAAATTT | | |
| | TCACCATTTACG | | |
| | AACGATAGCGCT | | |
| | (SEQ ID NO: 15) | | |
| 2560_pUC- | actagtAGTGACTG | Optimal 5′ UTR sequence for | Mohamad-Gabriel |
| ccTEV-A101 | ACTAGGATCTGG | mRNA stability and half-life | Alameh, Drew |
| 3′UTR | TTACCACTAAAC | from screens by Mohamad- | Weissman et al. |
| | CAGCCTCAAGAA | Gabriel Alameh, Drew | unpublished |
| | CACCCGAATGGA | Weissman et al. | |
| | GTCTCTAAGCTA | | |
| | CATAATACCAAC | | |
| | TTACACTTACAA | | |
| | AATGTTGTCCCC | | |
| | CAAAATGTAGCC | | |
| | ATTCGTATCTGC | | |
| | TCCTAATAAAAA | | |
| | GAAAGTTTCTTC | | |
| | ACATTCT (SEQ ID | | |
| | NO: 16) | | |
| poly A | AAAAAAAAAAA | Optimal poly A tail sequence | Jalkanen et al. Semin |
| (immediatly | AAAAAAAAAAA | for mRNA stability and half- | Cell Dev Biol. 34:24- |
| after 3′UTR) | AAAAAAAAAAA | life. | 32 (2014) |
| | AAAAAAAAAAA | | |
| | AAAAAAAAAAA | | |
| | AAAAAAAAAAA | | |
| | AAAAAAAAAAA | | |
| | AAAAAAAAAAA | | |
| | AAAAAAAAAAA | | |
| | AA (SEQ ID NO: | | |
| | 17) | | |
| mRNA codon | Reverse translate | Codon optimization is | Leppek et al. Nature |
| optimization | protein amino acid | performed as follow: amino | Communications |
| | sequence to optimal | acid sequence is reverse | 13: 1536 (2022) |
| | codons (see | translated into an DNA | |
| | ″Purpose″ for | sequence using a modified | |
| | description) | mamalian codon usage table. | |
| | | The table inceases both the | |
| | | CIA and the GC content of | |
| | | the mRNA. The reverse | |
| | | translated sequence (or | |
| | | mRNA sequence) is modeled | |
| | | into mFold and Delta H/Delta | |
| | | G computed, and the | |
| | | sequence with the lowest | |
| | | free energy is selected. In | |
| | | some cases, the codons can | |
| | | be replaced in specific | |
| | | locations to relax the | |
| | | tridimentional | |
| | | structure of the optimized | |
| | | mRNA. The sequnece is then | |
| | | cloned between the 5′UTR | |
| | | and 3′UTR above. | |

The modified proteins can have properties that are different than the unmodified protein from which the modified proteins are derived. In various embodiments, the modified proteins can be more likely to retain a prefusion conformation and less likely to assume a postfusion conformation as compared to the unmodified protein from which the modified protein is derived. In embodiments, the modified protein can be more stable than the unmodified protein. In embodiments, the modified protein can be produced in higher yields than the unmodified protein under similar experimental conditions. In some embodiments, increased stability can result in improved protein yield in recombinant expression systems. In some embodiments, the modified proteins can have longer half-life in the body of an individual who has been administered the protein (e.g., as a vaccine).

In embodiments, the modified proteins can substantially retain the immunogenicity or antigenicity of the unmodified protein from which the modified protein is derived. In examples, retention of immunogenicity can be determined by binding of various antibodies to the modified protein as compared to the unmodified protein (i.e., antibody binding profile). In some examples, antibody binding profiles can be obtained through use of combinations of antibodies that span multiple epitopes on the proteins. In some examples, neutralizing antibodies and/or non-neutralizing antibodies can be used.

In some examples, a nonlimiting list of antibodies that can be used to develop an antibody binding profile can include DH270 UCA3 (Saunders, Kevin O., et al. “Targeted selection of HIV-specific antibody mutations by engineering B cell maturation.” Science 366.6470 (2019): eaay7199), DH270.6 (Bonsignori, Mattia, et al. “Staged induction of HIV-1 glycan-dependent broadly neutralizing antibodies.” Science translational medicine 9.381 (2017): eaai7514.), PGT125 and PGR128 (Walker, Laura M., et al. “Broad neutralization coverage of HIV by multiple highly potent antibodies.” Nature 477.7365 (2011): 466-470), 2G12 (Trkola, Alexandra, et al. “Human monoclonal antibody 2G12 defines a distinctive neutralization epitope on the gp120 glycoprotein of human immunodeficiency virus type 1.” Journal of virology 70.2 (1996): 1100-1108), PGT151 (Falkowska, Emilia, et al. “Broadly neutralizing HIV antibodies define a glycan-dependent epitope on the prefusion conformation of gp41 on cleaved envelope trimers.” Immunity 40.5 (2014): 657-668), N6 (Huang, Jinghe, et al. “Identification of a CD4-binding-site antibody to HIV that evolved near-pan neutralization breadth.” Immunity 45.5 (2016): 1108-1121), F105 (Posner, Marshall R., et al. “Neutralization of HIV-1 by F105, a human monoclonal antibody to the CD4 binding site of gp120.” Journal of acquired immune deficiency syndromes 6.1 (1993): 7-14, A32 (Liao, Hua-Xin, et al. “Immunogenicity of constrained monoclonal antibody A32-human immunodeficiency virus (HIV) Env gp120 complexes compared to that of recombinant HIV type 1 gp120 envelope glycoproteins.” Journal of virology 78.10 (2004): 5270-5278), F93F (Moody, M. Anthony, et al. “Anti-phospholipid human monoclonal antibodies inhibit CCR5-tropic HIV-1 and induce β-chemokines.” Journal of Experimental Medicine 207.4 (2010): 763-776), 17B (Zhang, Wentao, et al. “Antibody 17b binding at the coreceptor site weakens the kinetics of the interaction of envelope glycoprotein gp120 with CD4.” Biochemistry 40.6 (2001): 1662-1670), 19B (Moore, John P., et al. “A human monoclonal antibody to a complex epitope in the V3 region of gp120 of human immunodeficiency virus type 1 has broad reactivity within and outside clade B.” Journal of virology 69.1 (1995): 122-130.), and combinations thereof.

In some embodiments, retention of immunogenicity can be determined by modeling proteins structures obtained using cryogenic electronic microscopy (cryo-EM), modeling the structures obtained from the method, and comparing data from the models (e.g., modified protein versus unmodified protein). In some embodiments, structural modeling of the proteins can be performed using data from cryo-EM to calculate a three-dimensional map of the protein. A model of the corresponding protein can be constructed using sequence information, stereochemical bond lengths and angles, and the three-dimensional map itself. The coordinates obtained from the model can be compared to coordinates from other models using, for example, root-mean-square-distance (RMSD), which calculates structural similarities between the two proteins based on positions of the paired C-alpha backbond carbons for each amino acid (e.g., Carugo, Oliviero, and Sándor Pongor. “A normalized root-mean-spuare distance for comparing protein three-dimensional structures.” Protein science 10.7 (2001): 1470-1473).

In an embodiment, the Env protein containing the 2P mutations (i.e., CH848 10.17DT DS-SOSIP-2P Env) was compared to an Env protein not containing the 2P mutations (i.e., CH848 10.17DT DS-SOSIP) and the RMSD was 0.757 Å over 1,253 C alpha atoms. In some embodiments, comparison of an Env that has 2P mutations with a protein that does not have 2P mutations can be less than about 2.0, 1.9, 1.8, 1.7, 1.6, 1.5, 1.4, 1.3, 1.2, 1.1, 1.0, 0.9, 0.8, 0.7, 0.6 or 0.5 Å.

### Vaccine Compositions

Herein, vaccine compositions refer to compositions of the modified proteins described herein suitable for administration to an individual for the purpose of prophylactically or therapeutically protecting the individual against, for example, an HIV-1, HIV-2 or SIV infection. Generally, the vaccine compositions contain prophylactically- or therapeutically-effective amounts of the modified proteins. In some embodiments, the vaccine compositions can include pharmaceutically acceptable carriers, diluents or excipients. The vaccine compositions can be administered to an individual by various routes, including oral, subcutaneous, parenteral (such as, intravenous, intraperitoneal), intramuscular, rectal, epidural, intratracheal, intranasal, dermal, vaginal, buccal, ocular and pulmonary administration.

### Methods

Disclosed are methods for making the modified proteins described herein. In some embodiments of the methods, amino acids within the proteins are selected for amino acid substitution based on the position and/or likely function of the amino acid in the protein. For type I fusion proteins that can change conformation from a prefusion form to a postfusion form (e.g., HIV and SIV Env proteins), amino acids that may play a role in the conformational change can be selected. In some embodiments, amino acid positions can be selected that, if substituted with a different amino acid, can decrease the probability that a prefusion conformation of the Env protein will change to a postfusion conformation. In some embodiments, these amino acid positions can be in flexible loops in the protein. In some examples, amino acids within the protein can be randomly or systematically substituted (e.g., a library of proteins can be made, each protein within the library have one or more amino acid substitutions).

In some embodiments, the substituting amino acid (i.e., the amino acid that replaces the amino acid in the unmodified protein) can prevent or decrease the probability that the protein, when in the prefusion conformation will transition to the postfusion conformation (e.g., inhibit the protein from forming an elongated alpha helix). In some embodiments, the substituting amino acid can have a lower configurational entropy than the amino acid that is substituted. In some embodiments, the substituting amino acid can be proline.

Generally, after the above-described amino acid substitutions are made (e.g., selected amino acids substituted or random amino acids substituted), the modified proteins containing the amino acid substitutions can be tested. In some embodiments, the ability of the modified proteins to be produced at high yields in, for example, recombinant protein expression systems, can be tested. In some embodiments, modified proteins produced at higher yield than unmodified proteins, under similar experimental conditions, are selected. In some embodiments, these high-yield modified proteins can be proteins with improved stability. Protein stability can also be tested more directly (rather than inferred from yields). For example, the ability of a modified protein to withstand repeated cycles of freeze and thaw can be tested. In some examples, thermostability of a modified protein can be determined (see Example 5). Other methods are known for investigating protein stability under various conditions, and for comparing stability of multiple proteins under different conditions. In some embodiments, the propensity of a modified protein that is in a prefusion conformation to assume a postfusion conformation can be tested.

In some embodiments, single substituted amino acids from different modified proteins that have increased yield, increases stability, decreased ability to form a postfusion conformation, as described above, can be combined in a single protein to test for additive, synergistic, or even antagonistic effects of the combined substitutions on the protein.

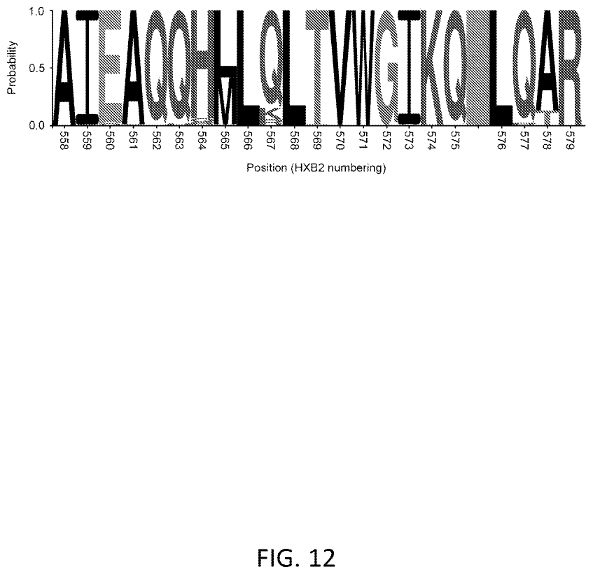

In some embodiments, amino acid positions to be substituted in a protein can be determined by referencing related proteins. For example, as discussed in Example 5 and illustrated in FIG. 12, Leu568 and Thr569 are 99.8% and 98.9% conserved among the 6,599 HIV-1 and SIVcpz Env sequences curated in the LANL HIV database (calculated using the AnalyzeAlign tool within the database). Therefore, in some embodiments, in proteins that have a threonine at position 568, a leucine at position 569, or a threonine at position 568 and a leucine at position 569, position 568 and/or 569 can be substituted with another amino acid. In some embodiments, the substituting amino acid can be proline.

In some embodiments, the amino acids at positions 568, 569, or 568 and 569 in an HIV Env can be identified and substituted with another amino acid. In some embodiments, the substituting aminoacid can be proline.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Single Mutants

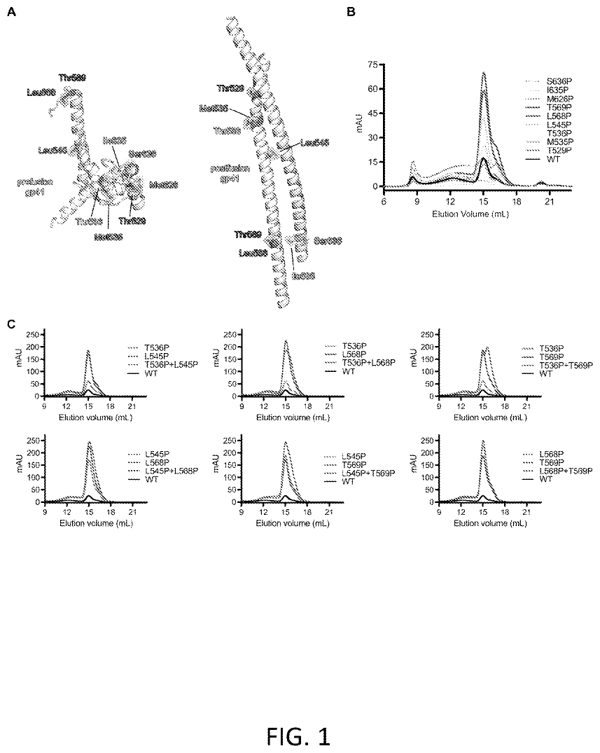

To enhance the yield of recombinantly expressed prefusion Env, we designed a series of single proline substitutions throughout the truncated gp41 subunit. Based on structural analysis of both the prefusion and postfusion conformations of gp41, nine positions were selected specifically to disfavor the formation of the elongated alpha helices which are characteristic of class I fusion proteins in the postfusion state (FIG. 1A). These substitutions were then made in the CH848 10.17DT Env, a clade C Envelope that has been modified to engage the UCA (unmutated common ancestor) from the DH270 V3 glycan bnAb lineage (27). This Env was expressed using a chimeric gp41 subunit from BG505 and was stabilized with both SOSIP.664 and DS mutations (CH848 10.17DT DS-SOSIP) (17, 24, 28). Purification of these constructs after transient transfection revealed that several of the individual proline substitutions had a substantial effect on Env yield (FIG. 1B). Envs with proline substitutions at positions 536, 545, 568 or 569 (HXB2 numbering) in particular showed a marked increase in the yield of prefusion trimer relative to the unmutated construct, therefore these four mutations were down selected for further analysis.

### Example 2—Double Mutants

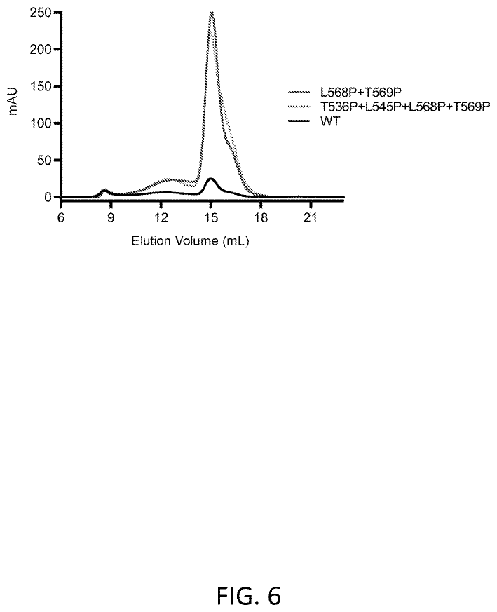

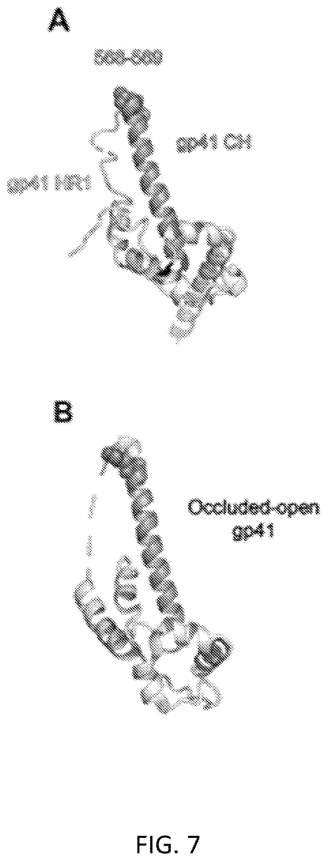

Individual proline mutations at positions 536, 545, 568 or 569 were combined to form double-mutants in an effort to determine whether these substitutions were capable of functioning synergistically (FIG. 1C). Modest improvements in yield over the single substitutions could be observed after combining substitutions 536+545 or 568+569, but other combinations resulted in either decreased yield (536+568) or the appearance of a prominent contaminating low-molecular-weight species which was readily detectable by size-exclusion chromatography (536+569, 545+568, 545+569). Similarly, combining proline substitutions at all four positions did not result in enhanced yield as compared to the double mutant at positions 568 and 569 (FIG. 6), which showed a ˜8-fold increase in prefusion trimer relative to CH848 10.17DT DS-SOSIP. Therefore, the double mutant at positions 568 and 569 was selected for subsequent characterization and these mutations were termed “2P”. Generally the residues upstream of this helical capping position in gp41 cannot be clearly resolved during structural characterization, presumably due to a high degree of conformational flexibility. However, the recently reported structure of Env in the occluded-open conformation shows these amino acids forming a 5-residue helical extension of the central helix compared to what can normally be observed in the gp41 prefusion conformation (FIG. 7B suggesting that the 2P mutations may be stabilizing the prefusion conformation of gp41 by disfavoring the sampling of this early intermediate (30).

### Example 3—Reverting I559P

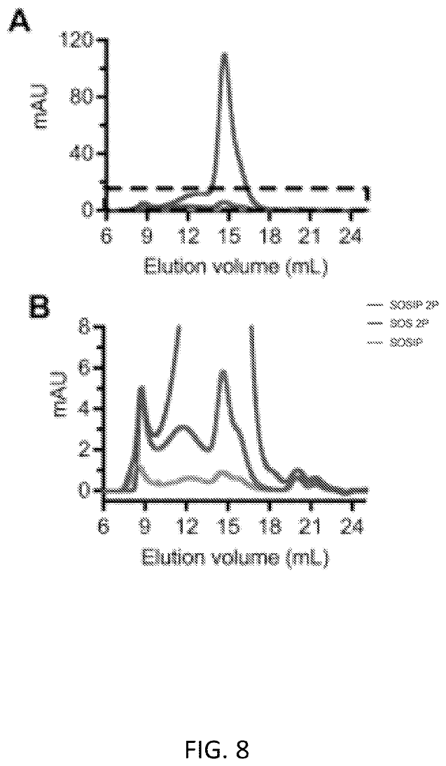

We next generated an “SOS 2P” Env by reverting the I559P mutation to investigate whether the new 2P mutations were capable of increasing the yield of prefusion Env in the absence of the stabilizing effect of the IP mutation. The SOS 2P construct yielded only slightly more prefusion trimer than the SOSIP Env, while the dramatic boost in yield that was observed previously could only be recapitulated when both the IP and 2P mutations were present (FIG. 8A-B).

### Example 4—Antigenic Landscape

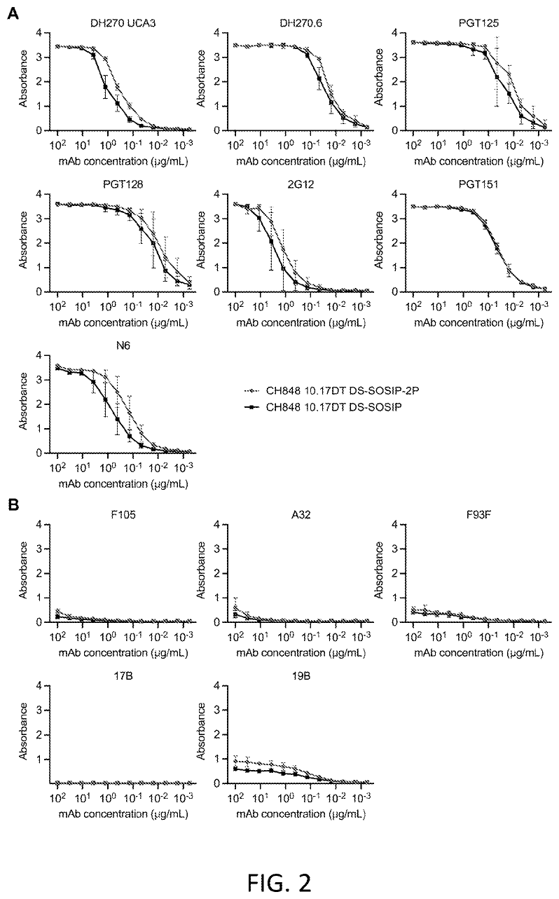

To determine whether the 2P mutations altered the antigenic landscape of the Env ectodomain, we performed an ELISA to compare the binding profiles of CH848 10.17DT DS-SOSIP and CH848 10.17DT DS-SOSIP-2P against a panel of well-characterized monoclonal antibodies (mAbs), described earlier in this application. Both neutralizing and non-neutralizing mAbs spanning multiple epitopes were included to comprehensively evaluate what effect the 2P mutations might have on Env folding (FIG. 2A, neutralizing antibodies; FIG. 2B, non-neutralizing antibodies). Overall, the binding characteristics exhibited by the two Envs were extremely similar, although the 2P-stabilized construct appeared to bind to some neutralizing mAbs directed against the V3 glycan epitope (DH270 UCA3) and the CD4-binding site (N6) with slightly higher affinity (FIG. 2A).

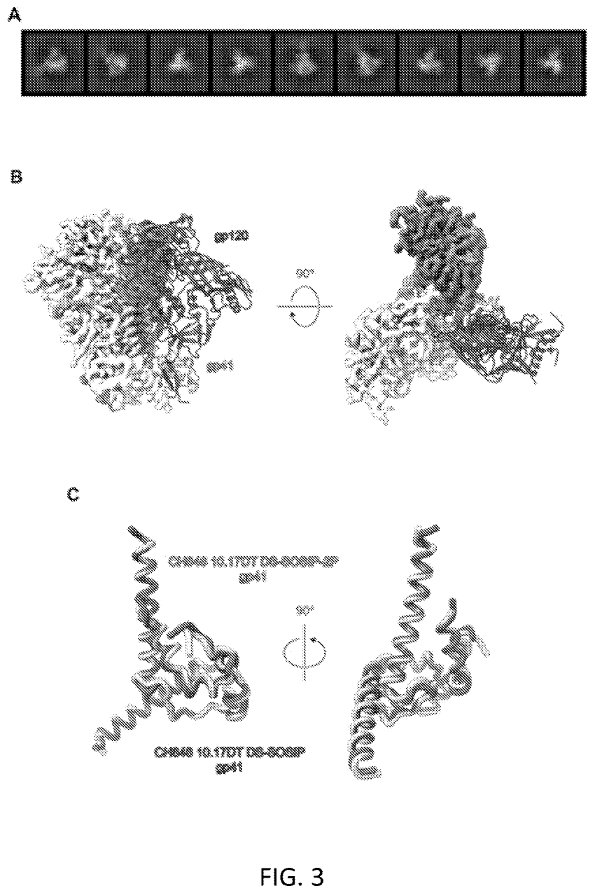

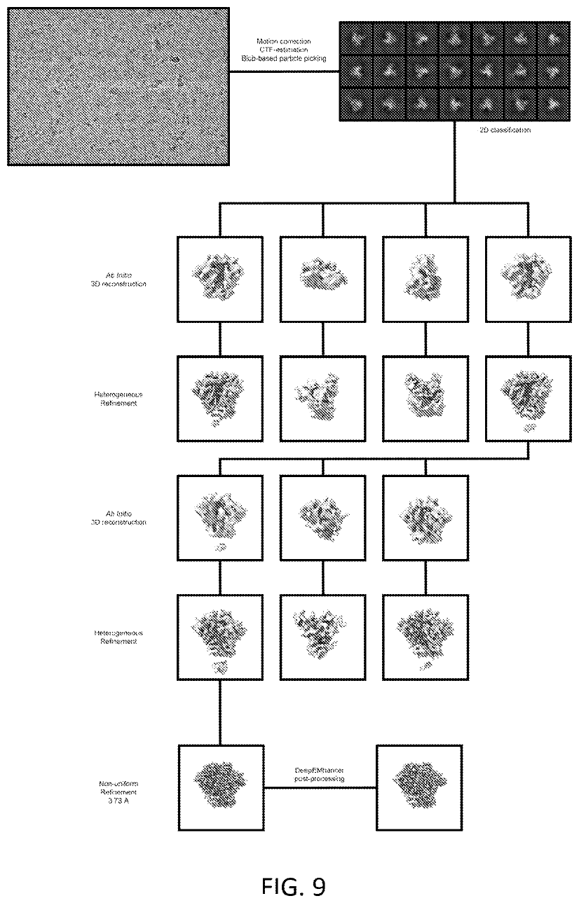

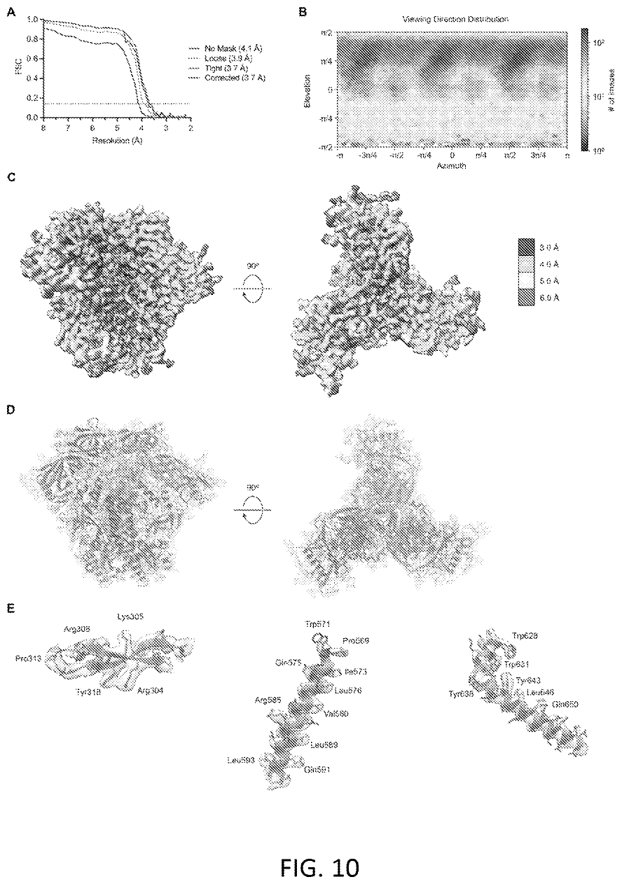

To further validate that the newly introduced 2P mutations did not disrupt the overall folding of the gp41 subunit, we determined the cryo-EM structure of the CH848 10.17DT DS-SOSIP-2P Env to a resolution of 3.73 Å (FIG. 3A-B, FIG. 9, FIG. 10A-E, Table 2).

TABLE 2
Cryo-EM collection and refinement statistics
Cryo-EM Data Collection

| Microscope | Titan Krios |
|---|---|
| Voltage (kV) | 300 |
| Detector | Gatan K3 |
| Pixel size (Å/pix) | 1.08 |
| Exposure rate (e−/pix/sec) | |
| Frames per exposure | |
| Exposure (e−/Å²) | 61 |
| Defocus range (αM) | |
| Micrographs used | 5,657 |
| Total particles extracted | 5,780,212 |

3D Reconstruction Statistics

| | |
|---|---|
| Particles | 111,026 |

| | | |
|---|---|---|
| Symmetry | C3 | C1 |
| Resolution (Å) by FSC | | |
| Unmasked 0.5 | 4.46 | 5.16 |
| Masked 0.5 | 4.10 | 4.25 |
| Unmasked 0.143 | 4.10 | 4.30 |
| Masked 0.143 | 3.73 | 3.95 |
| EMDB ID | | |

Model Refinement and Validation Statistics

| | |
|---|---|
| Composition | |
| Amino Acids | 1692 |
| Ligands (NAG) | 63 |
| RMSD Bonds | |
| Length (Å) | 0.01 |
| Angles (°) | 1.87 |
| Ramachandran plot | |
| Outliers (%) | 0 |
| Allowed (%) | 5.19 |
| Favored (%) | 94.81 |
| Rotamer outliers (%) | 0 |
| CD outliers (%) | 0 |
| Clash score | 3.25 |
| MolProbity score | 1.47 |
| EMRinger score | 3.17 |
| PDB ID | |

Our model spanned residues 32-664 and we were able to build N-linked glycans at 15 of the 25 putative sequons. There were several small, flexible loops in the gp120 subunit (59-66, 458-459, 400-411) that could not be confidently modeled, but overall the CH848 10.17DT DS-SOSIP-2P Env was nearly indistinguishable from the structures of previously reported CH848 10.17DT Envs which lack 2P stabilization (27). Like most other previously reported structures of gp41s in the prefusion conformation, the flexibility at the N-terminus of the central helix precluded us from observing both of our newly introduced proline substitutions, and only Pro569 could be confidently modeled into our reconstruction. However, the overall RMSD between our 2P-stabilized Env and a previously determined structure of CH848 10.17DT DS-SOSIP was only 0.757 Å over 1,253 Ca atoms (FIG. 3B), confirming that the 2P mutations are capable of enhancing the yield of recombinantly expressed Env ectodomain without altering the conformation of the SOSIP trimer.

### Example 5—Investigating Yields

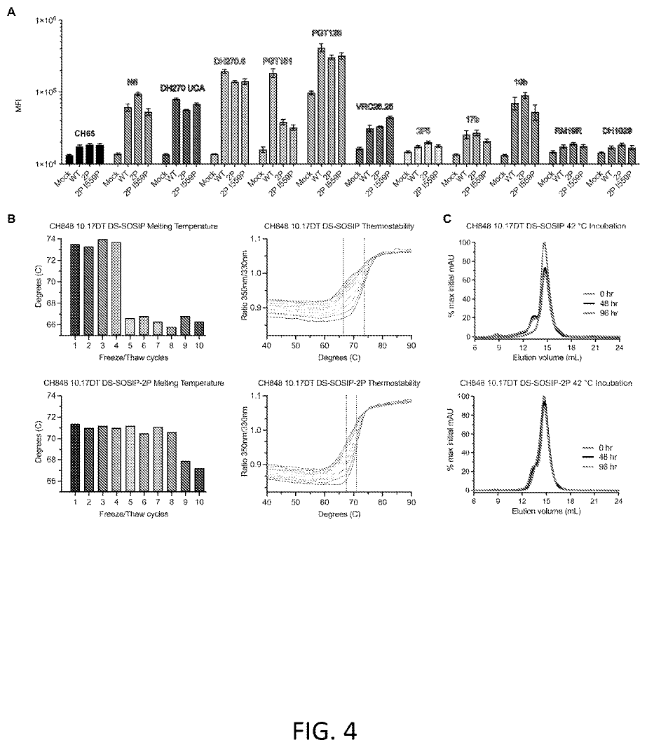

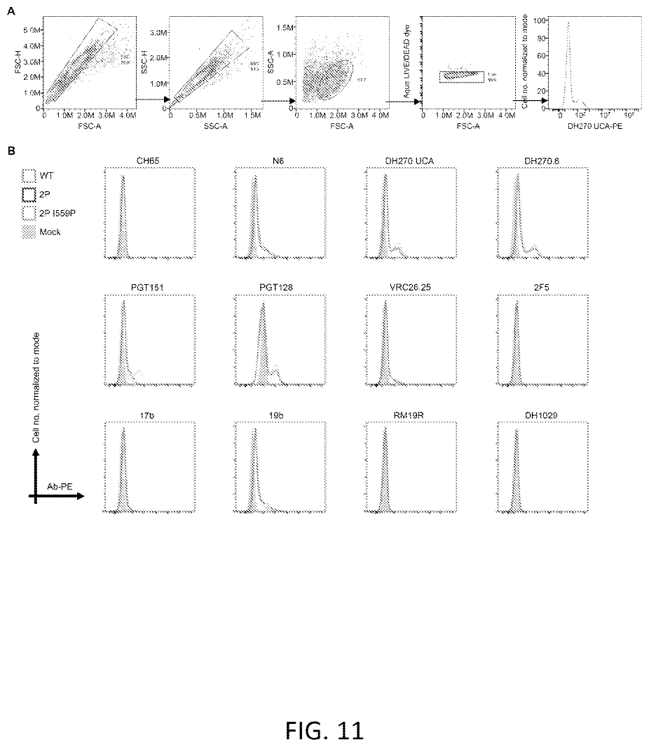

While not wanting to be held to a mechanism, the increase in the yield of prefusion trimer could be due to either an increase in Env expression or due to enhanced durability of the prefusion conformation upon proline-stabilization. To delineate between these two possibilities, we began by investigating what impact proline-stabilization would have on cell-surface expression of full-length Env. FreeStyle 293-F cells were transiently transfected with CH848 10.17DT gp160s containing either no proline mutations, 2P mutations, or 2P mutations in combination with the 1559P mutation. After 48 hours, the relative expression level of each transmembrane construct was estimated by flow cytometry after staining with a panel of monoclonal antibodies. Despite the dramatic differences in yield that could be observed when evaluating the recombinant expression of soluble ectodomains, no significant differences in surface expression levels of gp160 could be detected among the three constructs that were tested (FIG. 4A, FIG. 11A-B). The only clear difference between the three gp160s was a decrease in PGT151 binding in the 2P and the 2P+1559P constructs relative to the unstabilized gp160. However, this trend was not conserved throughout the rest of the antibody panel, nor was there a detectable difference between the 2P-stabilized and non-2P-stabilized Envs when testing PGT151 binding to soluble ectodomains (FIG. 2A).

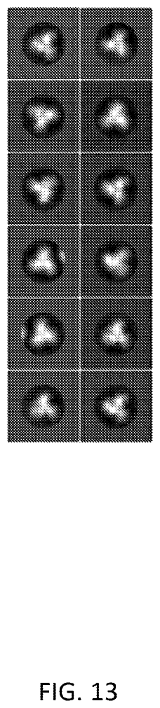

Based on these similarities in cell-surface expression levels, we next evaluated the durability of recombinantly expressed, proline-stabilized Env ectodomains. Aliquots of CH848 10.17DT DS-SOSIP and CH848 10.17DT DS-SOSIP-2P were subjected to ten rounds of rapid freezing and thawing. Thermostability measurements of both Envs were collected by differential scanning fluorimetry (DSF) during each round (FIG. 4B). The melting temperature (Tm) of CH848 10.17DT DS-SOSIP began at 73.6° C., a value that was maintained until after four freeze/thaw cycles, when it abruptly dropped to 66.4° C., where it remained throughout the rest of the experiment. The Tm of CH848 10.17DT DS-SOSIP-2P was initially measured to be 71.0° C., a value that was maintained until after eight freeze/thaw cycles, when the Tm dropped to 67.5° C. To further investigate this phenomenon, we performed a forced degradation assay in which aliquots of CH848 10.17DT DS-SOSIP and CH848 10.17DT DS-SOSIP-2P were incubated at 42° C. for either 48 or 96 hours. The integrity of the incubated trimers was then evaluated by size-exclusion chromatography (FIG. 4C). After 48 hours at 42° C., we observed the appearance of a high-molecular weight aggregate peak in the non-2P-stabilized sample. The emergence of this peak corresponded with a decrease in the amount of properly folded trimer (FIG. 13), centered around 14.8 mL of elution volume. A similar, albeit less prominent, peak was not observed in the 2P-stabilized sample until after 96 hours of incubation at 42° C., and the decrease of the peak at 14.8 mL was less pronounced, as measured by quantifying the area under the curve (Table 3).

TABLE 3
Area under the cure measurements
for SEC forced degradation assay

| | | AUC | | |
|---|---|---|---|---|
| | Incubation | 0.00-13.80 | 13.80-16.25 | 16.25-25.00 |
| Env | Period (hr) | mL | mL | mL |
| CH848 10.17DT | 0 | 8.41% | 88.31% | 3.28% |
| DS-SOSIP | 48 | 25.82% | 69.63% | 4.55% |
| | 96 | 26.37% | 66.69% | 6.95% |
| CH848 10.17DT | 0 | 11.45% | 85.26% | 3.29% |
| DS-SOSIP-2P | 48 | 16.07% | 80.18% | 3.75% |
| | 96 | 20.10% | 75.37% | 4.52% |

These data, in conjunction with our evaluation of cell-surface expression levels, indicate that proline-stabilization of the gp41 subunit has no impact on the level of protein expression, and that observed increases in the yield of recombinant ectodomain after proline-stabilization are instead due to the increased durability of the prefusion conformation of Env.

### Example 5—Conservation Among HIV Envs

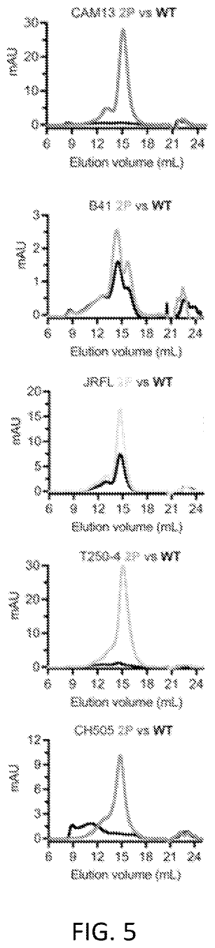

Leu568 and Thr569 are 99.8% and 98.9% conserved among the 6,599 Env sequences curated in the LANL HIV database (FIG. 12), possibly reflecting their functional importance during the transition from prefusion-to-postfusion and their insulation from antibody-mediated selective pressure. This high degree of sequence conservation at positions 568 and 569 prompted us to evaluate whether our 2P stabilization strategy might also be effective outside of the context of the CH848 10.17DT Env. A panel of evolutionarily diverse Envs (Table 4) was selected and the yield of recombinantly expressed prefusion trimer after transient transfection of a 2P-stabilized construct was compared to that of a non-2P-stabilized construct (FIG. 5).

TABLE 4
Env panel characteristics

| | | | | Additional |
|---|---|---|---|---|
| Abbre- | | Clade/ | GenBank | Mutations |
| viated | | Sub | Accession | (excluding |
| Name | Full Name | group | Number | 2P) |
| CH848 | CH048.3.d0949.10.17 | C | KX217749 | SOSIP.664¹, |
| 10.17DT | | | | DS², DT |
| | | | | (N133D + |
| | | | | N138T), |
| | | | | chimeric |
| | | | | BG505 |
| | | | | gp41³ |
| CAM13 | SIVcpzCAM13 | Siv | AY 169968 | SOSIP.664, |
| | | | | DS, Q171K |
| B41 | 9032-08.A1.4685 | B | EU5761 14.1 | SOSIP.664 |
| JRFL | JRFL | B | U63632.1 | SOSIPv6⁴ |
| T250-4 | T250-4 | 02_AG | MW507842 | SOSIP.664, |
| | | | | DS |
| CH505 | CH505w24 | C | — | SOSIP.664, |
| | | | | DS F14⁵ |

¹SOSIP.664 = A501C + T605C + I559P, R6 optimization of furin cleavage site, truncation after residue 664
²DS = 1201C + A433C
³Chimeric BG505 gp41 = Beginning at position 490, residues from BG505 Env were substituted for the native Env sequence
⁴SOSIPPv6 = SOSIP.664 + E64K + A316W + A73C + A561C + E49C + L555C
⁵F14 = V68I + A204V + V208L + V255L

Overall, 2P-stabilization improved the yield of Envs from HIV-1 clades B and C, subgroup 02_AG, and SIV. The degree to which 2P-stabilization enhanced Env yield was variable for the constructs that were evaluated, ranging from ˜2-fold increases in JRFL and B41 Envs to dramatic increases in the yields of CAM13, T250-4 and CH505 Envs, for which a fold-increase could not be reliably calculated due to an inability to purify the non-2P-stabilized construct. These findings suggest that 2P-stabilization is a broadly applicable approach to stabilizing the prefusion conformation of Env, albeit to varying degrees depending on the Env variant in question. Furthermore, the 2P mutations are compatible for addition to a variety of alternative Env stabilization strategies, including the SOSIP, DS and F14 mutations (Table 4).

### Example 6—Materials and Methods

### Protein Production and Purification

Plasmids encoding for Env ectodomains (CH848 10.17DT DS-SOSIP, CH848 10.17DT DS-SOS-2P, CH848 10.17DT DS-SOSIP-2P, CAM13 Q171K DS-SOSIP, CAM13 Q171K DS-SOSIP-2P, B41 SOSIP, B41 SOSIP-2P, JRFL SOSIPV6, JRFL SOSIPV6-2P, T250-4 DS-SOSIP, T250-4 DS-SOSIP-2P, CH505w24 F14 DS-SOSIP and CH505w24 F14 DS-SOSIP-2P) were mixed with a plasmid encoding furin at a ratio of 4:1. All Env constructs contained a C-terminal HRV3C cleavage site, a TwinStrepTag and an 8×HisTag (SEQ ID NO: 18). These plasmid mixtures were transfected into FreeStyle 293-F cells (Thermo Fisher) using polyethylenimine. Transfected supernatants were harvested and filtered five days post-transfection. Because CH848 Envs do not bind to PGT145, all CH848 constructs were purified by StrepTactin resin (IBA). All other Envs were purified by PGT145 affinity chromatography, as described previously (19). After affinity chromatography, Envs were further purified by size-exclusion chromatography using a Superose 6 Increase 10/300 GL column (Cytiva) in 2 mM Tris pH 8.0, 200 mM NaCl, 0.02% NaN3.

Plasmids encoding the heavy and light chains of mAbs used for ELISA or flow cytometric analysis (CH65, N6, DH270 UCA, DH270.6, PGT151, PGT128, VRC26.25, 2F5, 17b, 19b, RM19R, DH1029, 2G12, F93F, PGT125, F105, A32 and CH58) were combined at a ratio of 1:1 and used to transiently transfect Expi293 cells (Thermo Fisher) with ExpiFectamine (Thermo Fisher). Transfected supernatants were harvested and filtered five days post-transfection and antibodies were purified using Protein A resin (Thermo Fisher). Eluted antibodies were then buffer exchanged into PBS.

### Elisa

Env protein containing a C-terminal StrepTag was bound in wells of 384-well plates, which were previously coated with streptavidin (Thermo Fisher Scientific) at 2 μg/ml and blocked with PBS containing 4% (w/v) whey protein, 15% normal goat serum, 0.5% Tween-20, and 0.05% sodium azide. Proteins were incubated at room temperature for 1 hour, washed with PBS and 0.1% Tween-20, then mAbs were added in serial dilutions beginning at 100 μg/ml. Antibodies were incubated at room temperature for 1 hour, washed, and binding detected with goat anti-human HRP (Jackson ImmunoResearch) and TMB substrate (Sera Care Life Sciences).

### Cryo-EM Sample Preparation and Data Collection

Purified CH848 10.17DT DS-SOSIP-2P was diluted to a final concentration of 1.8 mg/mL in 10 mM Tris pH 8.0. To prevent interaction of the trimer with the air-water interface during vitrification, the sample was incubated in 0.085 mM n-dodecyl β-D-maltoside (DDM). A 3.5 μL drop of protein was deposited on a Quantifoil-1.2/1.3 grid (Electron Microscopy Sciences) that had been glow discharged for 10 seconds using an easiGlow Glow Discharge Cleaning System (PELCO). After a 30 second incubation in >95% humidity, excess protein was blotted away for 2.5 seconds before being plunge frozen into liquid ethane using a Leica EM GP2 plunge freezer (Leica Microsystems). Frozen grids were imaged using a Titan Krios (Thermo Fisher) equipped with a K3 detector (Gatan). Data were collected using the Gatan Latitude software.

### Cryo-EM Data Analysis and Refinement

Movies were imported into cryoSPARC v3.3.1 (38) and aligned using patch-based motion correction. Patch-based CTF estimation was then performed before 5,780,212 particles were selected using a non-templated blob picking strategy. Junk particles were then removed by 2D classification, leaving a stack of 683,406 particles that were subjected to iterative rounds of ab initio volume calculation and heterogeneous 3D classification, leaving a final stack of 111,026 particles. These particles yielded a 3.95 Å reconstruction after performing asymmetrical (C1) non-uniform refinement, with the resulting map exhibiting C3 symmetry. Non-uniform refinement (39) was then performed again using the same particle stack and applying C3 symmetry which improved the resolution of the resulting reconstruction to 3.73 Å. This map was then subjected to post-processing using DeepEMhancer (40). A full description of the cryo-EM data processing workflow can be found in FIG. 9. Chain A, D, E, H, I and 1 from PDB ID: 6UM7 were used as a starting model that was docked into the sharpened map and re-modeled through iterative rounds of building and refinement in Coot (41), PHENIX (42) and ISOLDE (43).

### Gp160 Cell-Surface Expression and Characterization

293-F cells (ThermoFisher, cat #R79007) were diluted to 1.25×10⁶ cells/mL and seeded to 12-well plates 2-3 hours before transfection. Transient transfection of DNA plasmids was performed with jetPRIME transfection reagent (Polyplus, cat #101000046) following the manufacturer's instruction. Transfected cells were cultured in an incubator at 37° C. with 8% CO2 and shaking at 125 rpm for 24 hours before flow cytometry staining.

24 hours after transfection, 293-F cells were harvested, counted and then were rinsed with 1% BSA/PBS and pelleted at 500 g for 5 minutes. Next, cells were resuspended to a density of 1×10⁶ cells/mL in 1% BSA/PBS and 50,000 cells were aliquoted to each well of a U-bottom 96-well plates. An equal volume of recombinant anti-HIV-1 Env antibodies at 4 μg/mL were added to cells to reach a working concentration of 2 μg/mL. Antibodies were incubated with cells at 4° C. for 30 minutes. Cells were then washed once with 150 μL 1% BSA/PBS and then incubated with 50 μL Goat anti-Human IgG Fc secondary antibody PE (ThermoFisher, cat #12-4998-82) at a final concentration of 2.5 μg/mL in 1% BSA/PBS. After a 30 minute incubation at 4° C. protected from light, cells were washed once with 1×PBS and incubated with 100 μL LIVE/DEAD Aqua dead cell stain (L34966, 1:1000 in PBS) for 20 minutes at room temperature, protected from light. Next, cells were washed once with 100 μL 1% BSA/BSA, then resuspended in 50 μL 1% BSA 2 mM EDTA 1% PFA in PBS. Flow cytometric data were acquired on an iQue 3 high-throughput flow cytometry system (Sartorius). Data were analyzed using FlowJo v10 (FlowJo).

### Freeze/Thaw Thermostability Analysis

Purified CH848 10.17DT DS-SOSIP and CH848 10.17DT DS-SOSIP-2P were diluted to 0.2 mg/mL in 2 mM Tris pH 8.0, 200 mM NaCl, 0.02% NaN3. Samples were rapidly frozen in liquid nitrogen and thawed by incubation at 30 □C for 5 minutes. Thermostability measurements were collected using a Tycho NT.6 by increasing the temperature from 35° C. to 95° C. at a rate of 30.0° C./minute. Tm was determined as the inflection temperature using the Tycho Nanotemper data processing software.

### Forced Degradation Assay

250 μg aliquots of purified CH848 10.17DT DS-SOSIP or CH848 10.17DT DS-SOSIP-2P were incubated at 42° C. for 0 hours, 48 hours or 96 hours. Aliquots were then run over a Superose 6 Increase 10/300 GL column (Cytiva Life Sciences) to evaluate Env integrity.

### Negative Stain Electron Microscopy

CH848 10.17DT DS-SOSIP-2P was diluted to 0.2 mg/mL with buffer containing 20 mM HEPES pH 7.4, 150 mM NaCl, 8 mM glutaraldehyde and 5 g/dL glycerol. After 5 minutes of incubation, excess glutaraldehyde was quenched by the addition 1 M TRIS pH 8.0 for a final concentration of 80 mM TRIS and incubated for an additional 5 minutes. Quenched sample was applied to a glow-discharged, carbon-coated EM grid for 8-10 seconds, blotted, and stained with 2 g/dL uranyl formate for 1 minute before being blotted and allowed to air-dry. The stained grid was examined on a Philips EM420 electron microscope operating at 120 kV and a nominal magnification of 49,000×. 12 images were collected on a 76 Mpix CCD camera at 2.4 Å/pix. Images were analyzed and 2D class averaging was performed using standard protocols within Relion 3.0 (Zivanov, Jasenko, et al. “New tools for automated high-resolution cryo-EM structure determination in RELION-3.” elife 7 (2018): e42166).

### REFERENCES

1. Centers for Disease C. 1981. Kaposi's sarcoma and Pneumocystis pneumonia among homosexual men—New York City and California. MMWR Morb Mortal Wkly Rep 30:305-8.
2. Eisinger R W, Fauci A S. 2018. Ending the HIV/AIDS Pandemic (1). Emerg Infect Dis 24:413-416.
3. Porter K, Johnson A M, Phillips A N, Darbyshire J H. 1999. The practical significance of potential biases in estimates of the AIDS incubation period distribution in the U K register of HIV seroconverters. AIDS 13:1943-51.
4. Grant R M, Lama J R, Anderson P L, McMahan V, Liu A Y, Vargas L, Goicochea P, Casapia M, Guanira-Carranza J V, Ramirez-Cardich M E, Montoya-Herrera O, Fernandez T, Veloso V G, Buchbinder S P, Chariyalertsak S, Schechter M, Bekker L G, Mayer K H, Kallas E G, Amico K R, Mulligan K, Bushman L R, Hance R J, Ganoza C, Defechereux P, Postle B, Wang F, McConnell J J, Zheng J H, Lee J, Rooney J F, Jaffe H S, Martinez A I, Burns D N, Glidden D V, iPrEx Study T. 2010. Preexposure chemoprophylaxis for HIV prevention in men who have sex with men. N Engl J Med 363:2587-99.
5. Palella F J, Jr., Delaney K M, Moorman A C, Loveless M O, Fuhrer J, Satten G A, Aschman D J, Holmberg S D. 1998. Declining morbidity and mortality among patients with advanced human immunodeficiency virus infection. HIV Outpatient Study Investigators. N Engl J Med 338:853-60.
6. Fauci A S. 2017. An HIV Vaccine Is Essential for Ending the HIV/AIDS Pandemic. JAMA 318:1535-1536.
7. Allan J S, Coligan J E, Barin F, McLane M F, Sodroski J G, Rosen C A, Haseltine W A, Lee T H, Essex M. 1985. Major glycoprotein antigens that induce antibodies in AIDS patients are encoded by HTLV-III. Science 228:1091-4.
8. Willey R L, Bonifacino J S, Potts B J, Martin M A, Klausner R D. 1988. Biosynthesis, cleavage, and degradation of the human immunodeficiency virus 1 envelope glycoprotein gp160. Proc Natl Acad Sci USA 85: 9580-4.
9. Dalgleish A G, Beverley P C, Clapham P R, Crawford D H, Greaves M F, Weiss R A. 1984. The CD4 (T4) antigen is an essential component of the receptor for the AIDS retrovirus. Nature 312:763-7.
10. Klatzmann D, Champagne E, Chamaret S, Gruest J, Guetard D, Hercend T, Gluckman J C, Montagnier L. 1984. T-lymphocyte T4 molecule behaves as the receptor for human retrovirus LAV. Nature 312:767-8.
11. Alkhatib G, Combadiere C, Broder C C, Feng Y, Kennedy P E, Murphy P M, Berger E A. 1996. CCCKR5: a RANTES, MIP-1alpha, MIP-1beta receptor as a fusion cofactor for macrophage-tropic HIV-1. Science 272:1955-8.
12. Kowalski M, Potz J, Basiripour L, Dorfman T, Goh W C, Terwilliger E, Dayton A, Rosen C, Haseltine W, Sodroski J. 1987. Functional regions of the envelope glycoprotein of human immunodeficiency virus type 1. Science 237:1351-5.
13. Wei X, Decker J M, Wang S, Hui H, Kappes J C, Wu X, Salazar-Gonzalez J F, Salazar M G, Kilby J M, Saag M S, Komarova N L, Nowak M A, Hahn B H, Kwong P D, Shaw G M. 2003. Antibody neutralization and escape by HIV-1. Nature 422:307-12.
14. Roark R S, Li H, Williams W B, Chug H, Mason R D, Gorman J, Wang S, Lee F H, Rando J, Bonsignori M, Hwang K K, Saunders K O, Wiehe K, Moody M A, Hraber P T, Wagh K, Giorgi E E, Russell R M, Bibollet-Ruche F, Liu W, Connell J, Smith A G, DeVoto J, Murphy A I, Smith J, Ding W, Zhao C, Chohan N, Okumura M, Rosario C, Ding Y, Lindemuth E, Bauer A M, Bar K J, Ambrozak D, Chao C W, Chuang G Y, Geng H, Lin B C, Louder M K, Nguyen R, Zhang B, Lewis M G, Raymond D D, Doria-Rose N A, Schramm C A, Douek D C, Roederer M, Kepler T B, Kelsoe G, et al. 2021. Recapitulation of HIV-1 Env-antibody coevolution in macaques leading to neutralization breadth. Science 371.
15. Patel P H, Preston B D. 1994. Marked infidelity of human immunodeficiency virus type 1 reverse transcriptase at RNA and DNA template ends. Proc Natl Acad Sci USA 91:549-53.
16. Weissenhorn W, Wharton S A, Calder L J, Earl P L, Moss B, Aliprandis E, Skehel J J, Wiley D C. 1996. The ectodomain of HIV-1 env subunit gp41 forms a soluble, alpha-helical, rod-like oligomer in the absence of gp120 and the N-terminal fusion peptide. EMBO J 15:1507-14.
17. Sanders R W, Vesanen M, Schuelke N, Master A, Schiffner L, Kalyanaraman R, Paluch M, Berkhout B, Maddon P J, Olson W C, Lu M, Moore J P. 2002. Stabilization of the soluble, cleaved, trimeric form of the envelope glycoprotein complex of human immunodeficiency virus type 1. J Virol 76:8875-89.
18. Binley J M, Sanders R W, Clas B, Schuelke N, Master A, Guo Y, Kajumo F, Anselma D J, Maddon P J, Olson W C, Moore J P. 2000. A recombinant human immunodeficiency virus type 1 envelope glycoprotein complex stabilized by an intermolecular disulfide bond between the gp120 and gp41 subunits is an antigenic mimic of the trimeric virion-associated structure. J Virol 74:627-43.
19. de Taeye S W, Ozorowski G, Torrents de la Pena A, Guttman M, Julien J P, van den Kerkhof T L, Burger J A, Pritchard L K, Pugach P, Yasmeen A, Crampton J, Hu J, Bontjer I, Torres J L, Arendt H, DeStefano J, Koff W C, Schuitemaker H, Eggink D, Berkhout B, Dean H, LaBranche C, Crotty S, Crispin M, Montefiori D C, Klasse P J, Lee K K, Moore J P, Wilson I A, Ward A B, Sanders R W. 2015. Immunogenicity of Stabilized HIV-1 Envelope Trimers with Reduced Exposure of Non-neutralizing Epitopes. Cell 163:1702-15.
20. Del Moral-Sanchez I, Russell R A, Schermer E E, Cottrell C A, Allen J D, Torrents de la Pena A, LaBranche C C, Kumar S, Crispin M, Ward A B, Montefiori D C, Sattentau Q J, Sliepen K, Sanders R W. 2022. High thermostability improves neutralizing antibody responses induced by native-like HIV-1 envelope trimers. NPJ Vaccines 7:27.
21. Torrents de la Pena A, Julien J P, de Taeye S W, Garces F, Guttman M, Ozorowski G, Pritchard L K, Behrens A J, Go E P, Burger J A, Schermer E E, Sliepen K, Ketas T J, Pugach P, Yasmeen A, Cottrell C A, Torres J L, Vavourakis C D, van Gils M J, LaBranche C, Montefiori D C, Desaire H, Crispin M, Klasse P J, Lee K K, Moore J P, Ward A B, Wilson I A, Sanders R W. 2017. Improving the Immunogenicity of Native-like HIV-1 Envelope Trimers by Hyperstabilization. Cell Rep 20:1805-1817.
22. Henderson R, Lu M, Zhou Y, Mu Z, Parks R, Han Q, Hsu A L, Carter E, Blanchard S C, Edwards R J, Wiehe K, Saunders K O, Borgnia M J, Bartesaghi A, Mothes W, Haynes B F, Acharya P, Munir Alam S. 2020. Disruption of the HIV-1 Envelope allosteric network blocks CD4-induced rearrangements. Nat Commun 11:520.
23. Kong L, He L, de Val N, Vora N, Morris C D, Azadnia P, Sok D, Zhou B, Burton D R, Ward A B, Wilson I A, Zhu J. 2016. Uncleaved prefusion-optimized gp140 trimers derived from analysis of HIV-1 envelope metastability. Nat Commun 7:12040.
24. Kwon Y D, Pancera M, Acharya P, Georgiev I S, Crooks E T, Gorman J, Joyce M G, Guttman M, Ma X, Narpala S, Soto C, Terry D S, Yang Y, Zhou T, Ahlsen G, Bailer R T, Chambers M, Chuang G Y, Doria-Rose N A, Druz A, Hallen M A, Harned A, Kirys T, Louder M K, O'Dell S, Ofek G, Osawa K, Prabhakaran M, Sastry M, Stewart-Jones G B, Stuckey J, Thomas P V, Tittley T, Williams C, Zhang B, Zhao H, Zhou Z, Donald B R, Lee L K, Zolla-Pazner S, Baxa U, Schon A, Freire E, Shapiro L, Lee K K, Arthos J, Munro J B, Blanchard S C, Mothes W, Binley J M, et al. 2015. Crystal structure, conformational fixation and entry-related interactions of mature ligand-free HIV-1 Env. Nat Struct Mol Biol 22:522-31.
25. Rutten L, Lai Y T, Blokland S, Truan D, Bisschop I J M, Strokappe N M, Koornneef A, van Manen D, Chuang G Y, Farney S K, Schuitemaker H, Kwong P D, Langedijk J P M. 2018. A Universal Approach to Optimize the Folding and Stability of Prefusion-Closed HIV-1 Envelope Trimers. Cell Rep 23:584-595.
26. Leaman D P, Zwick M B. 2013. Increased functional stability and homogeneity of viral envelope spikes through directed evolution. PLOS Pathog 9: e1003184.
27. Saunders K O, Wiehe K, Tian M, Acharya P, Bradley T, Alam S M, Go E P, Scearce R, Sutherland L, Henderson R, Hsu A L, Borgnia M J, Chen H, Lu X, Wu N R, Watts B, Jiang C, Easterhoff D, Cheng H L, McGovern K, Waddicor P, Chapdelaine-Williams A, Eaton A, Zhang J, Rountree W, Verkoczy L, Tomai M, Lewis M G, Desaire H R, Edwards R J, Cain D W, Bonsignori M, Montefiori D, Alt F W, Haynes B F. 2019. Targeted selection of HIV-specific antibody mutations by engineering B cell maturation. Science 366.
28. Joyce M G, Georgiev I S, Yang Y, Druz A, Geng H, Chuang G Y, Kwon Y D, Pancera M, Rawi R, Sastry M, Stewart-Jones G B E, Zheng A, Zhou T, Choe M, Van Galen J G, Chen R E, Lees C R, Narpala S, Chambers M, Tsybovsky Y, Baxa U, McDermott A B, Mascola J R, Kwong P D. 2017. Soluble Prefusion Closed DS-SOSIP.664-Env Trimers of Diverse HIV-1 Strains. Cell Rep 21:2992-3002.
29. Pallesen J, Wang N, Corbett K S, Wrapp D, Kirchdoerfer R N, Turner H L, Cottrell C A, Becker M M, Wang L, Shi W, Kong W P, Andres E L, Kettenbach A N, Denison M R, Chappell J D, Graham B S, Ward A B, Mclellan J S. 2017. Immunogenicity and structures of a rationally designed prefusion MERS-COV spike antigen. Proc Natl Acad Sci USA 114: E7348-E7357.
30. Yang Z, Dam K A, Bridges M D, Hoffmann M A G, DeLaitsch A T, Gristick H B, Escolano A, Gautam R, Martin M A, Nussenzweig M C, Hubbell W L, Bjorkman P J. 2022. Neutralizing antibodies induced in immunized macaques recognize the CD4-binding site on an occluded-open HIV-1 envelope trimer. Nat Commun 13:732.
31. Hsieh C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, Mclellan J S. 2020. Structure-based design of prefusion-stabilized SARS-COV-2 spikes. Science 369:1501-1505.
32. Barouch D H, O'Brien K L, Simmons N L, King S L, Abbink P, Maxfield L F, Sun Y H, La Porte A, Riggs A M, Lynch D M, Clark S L, Backus K, Perry J R, Seaman M S, Carville A, Mansfield K G, Szinger J J, Fischer W, Muldoon M, Korber B. 2010. Mosaic HIV-1 vaccines expand the breadth and depth of cellular immune responses in rhesus monkeys. Nat Med 16:319-23.
33. Haynes B F, Kelsoe G, Harrison S C, Kepler T B. 2012. B-cell-lineage immunogen design in vaccine development with HIV-1 as a case study. Nat Biotechnol 30:423-33.
34. Rawi R, Rutten L, Lai Y T, Olia A S, Blokland S, Juraszek J, Shen C H, Tsy bovsky Y, Verardi R, Yang Y, Zhang B, Zhou T, Chuang G Y, Kwong P D, Langedijk J P M. 2020. Automated Design by Structure-Based Stabilization and Consensus Repair to Achieve Prefusion-Closed Envelope Trimers in a Wide Variety of HIV Strains. Cell Rep 33:108432.
35. Steichen J M, Kulp D W, Tokatlian T, Escolano A, Dosenovic P, Stanfield R L, McCoy L E, Ozorowski G, Hu X, Kalyuzhniy O, Briney B, Schiffner T, Garces F, Freund N T, Gitlin A D, Menis S, Georgeson E, Kubitz M, Adachi Y, Jones M, Mutafyan A A, Yun D S, Mayer C T, Ward A B, Burton D R, Wilson I A, Irvine D J, Nussenzweig M C, Schief W R. 2016. HIV Vaccine Design to Target Germline Precursors of Glycan-Dependent Broadly Neutralizing Antibodies. Immunity 45:483-496.
36. Feng Y, Tran K, Bale S, Kumar S, Guenaga J, Wilson R, de Val N, Arendt H, DeStefano J, Ward A B, Wyatt R T. 2016. Thermostability of Well-Ordered HIV Spikes Correlates with the Elicitation of Autologous Tier 2 Neutralizing Antibodies. PLOS Pathog 12: e1005767.
37. Guttman M, Cupo A, Julien J P, Sanders R W, Wilson I A, Moore J P, Lee K K. 2015. Antibody potency relates to the ability to recognize the closed, pre-fusion form of HIV Env. Nat Commun 6:6144.
38. Punjani A, Rubinstein J L, Fleet D J, Brubaker M A. 2017. cryoSPARC: algorithms for rapid unsupervised cryo-E M structure determination. Nat Methods 14:290-296.
39. Punjani A, Zhang H, Fleet D J. 2020. Non-uniform refinement: adaptive regularization improves single-particle cryo-E M reconstruction. Nat Methods 17:1214-1221.
40. Sanchez-Garcia R, Gomez-Blanco J, Cuervo A, Carazo J M, Sorzano C O S, Vargas J. 2021. DeepEMhancer: a deep learning solution for cryo-E M volume post-processing. Commun Biol 4:874.
41. Emsley P, Cowtan K. 2004. Coot: model-building tools for molecular graphics. Acta Crystallogr D Biol Crystallogr 60:2126-32.
42. Adams P D, Grosse-Kunstleve R W, Hung L W, Joerger T R, McCoy A J, Moriarty N W, Read R J, Sacchettini J C, Sauter N K, Terwilliger T C. 2002. PHENIX: building new software for automated crystallographic structure determination. Acta Crystallogr D Biol Crystallogr 58:1948-54.
43. Croll T I. 2018. ISOLDE: a physically realistic environment for model building into low-resolution electron-density maps. Acta Crystallogr D Struct Biol 74:519-530.

### EQUIVALENTS

Those skilled in the art will recognize, or be able to ascertain, using no more than routine experimentation, numerous equivalents to the specific substances and procedures described herein. Such equivalents are considered to be within the scope of this invention.

What is claimed:

1. A modified type I fusion protein from a retrovirus, comprising substituted amino acids at one or more of positions 536, 545, 568 and 569 (HXB2 numbering).

2. The modified type I fusion protein of claim 1, wherein the retrovirus comprises a lentivirus.

3. The modified type I fusion protein of claim 2, wherein the retrovirus comprises a human immunodeficiency virus (HIV).

4. The modified type I fusion protein of claim 3, wherein the HIV comprises HIV-1 or HIV-2.

5. The modified type I fusion protein of claim 4, wherein the HIV-1 comprises a Group M, N or O HIV-1.

6. The modified type I fusion protein of claim 5, wherein the HIV-1 comprises a Group M HIV-1 from subtype/clade A, B, C, D, F, G, H, J or K.

7. The modified type I fusion protein of claim 6, wherein the HIV-1 comprises a Group M HIV-1 from subtype/clade B or C.

8. The modified type I fusion protein of claim 2, wherein the retrovirus comprises a simian immunodeficiency virus (SIV).

9. The modified type I fusion protein of claim 2, comprising an envelope protein (Env).

10. The modified type I fusion protein of claim 9, wherein the Env protein comprises a gp160, a gp120 or a gp41.

11. The modified type I fusion protein of claim 9, wherein the Env protein comprises an ectodomain of the Env protein.

12. The modified type I fusion protein of claim 9, comprising the substituted amino acids at one of positions 536, 545, 568 and 569.

13. The modified type I fusion protein of claim 9, comprising the substituted amino acids at any two of positions 536, 545, 568 and 569.

14. The modified type I fusion protein of claim 9, comprising the substituted amino acids at any three of positions 536, 545, 568 and 569.

15. The modified type I fusion protein of claim 9, comprising the substituted amino acids at positions 536, 545, 568 and 569.

16. The modified type I fusion protein of claim 9, comprising the substituted amino acids at positions 536 and 545, 536 and 568, 536 and 569, 545 and 568, 545 and 569, or 568 and 569.

17. The modified type I fusion protein of claim 16, comprising the substituted amino acids at positions 536 and 545 or at positions 568 and 569.

18. The modified type I fusion protein of claim 17, comprising the substituted amino acids at positions 568 and 569.

19. The modified type I fusion protein of claim 9, wherein the substituted amino acids at one or more of positions 536, 545, 568 and 569 are the only substituted amino acids in the modified protein.

20. The modified type I fusion protein of claim 9, wherein the modified protein additionally comprises an I559P (IP) amino acid substitution.

21. The modified type I fusion protein of claim 9, wherein the modified protein additionally comprises a disulfide bond (SOS) linking a gp120 and gp41 subunit.

22. The modified type I fusion protein of claim 9, wherein the modified protein additionally comprises an IP amino acid substitution and an SOS linking a gp120 and gp41 subunit (SOSIP).

23. The modified type I fusion protein of claim 9, wherein the Env protein comprises the substituted amino acids at positions 568 and 569 and comprises a proline at position 559.

24. The modified type I fusion protein of any one of claims 9 and 12-23, wherein the modified protein additionally comprises mutations DS (1201C+A433C), DT (N133D+N138T), chimeric BG 505 gp41, Q171K, SOSIPv6, F14 (V68I+A204V+V208L+V255L) or combinations thereof.

25. The modified type I fusion protein of claim 1, wherein the substituted amino acids at the one or more positions comprise proline.

26. The modified type I fusion protein of claim 1, wherein the substituted amino acids at the one or more positions comprise glycine.

27. The modified type I fusion protein of claim 9, wherein the substituted amino acid at position 568 replaces leucine, at position 569 replaces threonine, or at position 568 replaces leucine and at position 569 replaces threonine.

28. The modified type I fusion protein of claim 1, wherein the modified protein substantially retains an antibody binding profile of an unmodified type I fusion protein from which the modified type I fusion protein is derived.

29. The modified type I fusion protein of claim 28, wherein the antibody binding profile comprises binding to the modified protein of neutralizing and non-neutralizing antibodies that span multiple epitopes.

30. The modified type I fusion protein of claim 28, wherein antibodies used to obtain the antibody binding profile comprise DH270 UCA3, DH270.6, PGT125, PGR128, 2G12, PGT151, N6, F105, A32, F93F, 17B, 19B, and combinations thereof.

31. The modified type I fusion protein of claim 1, wherein the modified protein substantially retains antigenicity of an unmodified type I fusion protein from which the modified type I fusion protein is derived.

32. The modified type I fusion protein of claim 9, wherein coordinates of the modified type I fusion protein compared with a similar or identical protein that does not contain the substituted amino acids at one or more of positions 536, 545, 568 and 569 yield a root-mean-square-distance of less than 1.5, 1.4, 1.3, 1.2, 1.1, 1.0, 0.9, 0.8 or 0.7 angstroms, over 1200 alpha atoms.

33. The modified type I fusion protein of claim 32, wherein the coordinates of the modified type I fusion protein and the identical protein that does not contain the substituted amino acids are obtained from structural models of the proteins derived from cryogenic electronic microscopy images of the proteins.

34. The modified type I fusion protein of claim 32, wherein the similar or identical protein that does not contain the substituted amino acids comprises CH848 10.17DT DS-SOSIP.

35. The modified type I fusion protein of claim 9, wherein the modified protein is expressed at higher amounts in expression studies, is more stable, in a prefusion conformation is less likely to form a postfusion conformation than an unmodified type I fusion protein from which the modified type I fusion protein is derived, and combinations thereof.

36. A modified HIV envelope protein (Env) comprising prolines at positions 559, 568 and 569.

37. A method for modifying an envelope protein (Env) from a retrovirus, comprising:
substituting one amino acid in an unmodified Env protein with a substituting amino acid to obtain a modified Env protein; and
determining a stability of the modified Env protein as compared to a stability of the unmodified Env protein.

38. The method of claim 37, wherein a position of the one amino acid substitution is selected to inhibit the modified Env protein from assuming a postfusion conformation.

39. The method of claim 37, wherein a position of the one amino acid substitution is selected to inhibit the modified Env protein from forming an elongated alpha helix.

40. The method of claim 37, wherein a position of the one amino acid substitution is in a flexible loop of the unmodified Env protein.

41. The method of claim 37, wherein the substituting amino acid has a lower configuration entropy than an amino acid it replaces.

42. The method of claim 37, wherein the modified protein has a lower configurational or conformational entropy than the unmodified protein.

43. The method of claim 37, wherein the Env protein from a retrovirus comprises an Env protein from HIV-1, HIV-2 or SIV.

44. The method of claim 43, wherein the substituted one amino acid is at position 536, 545, 568 or 569 of the unmodified Env protein (HXB2 numbering).

45. The method of claim 41, wherein the substituting amino acid comprises proline.

46. The method of claim 37, further comprising selecting a modified Env from the method that is more stable than the unmodified Env.

47. The method of claim 46, wherein a substituting amino acid position from a first modified Env protein is combined with a substituting amino acid position from a second modified Env protein to form a third modified Env protein having the substituted amino acids of the first and the second modified Env proteins.

48. The method of claim 47, further comprising selecting a third modified Env protein from the method that is more stable than either the first or the second modified Env protein.

49. The method of claim 37, wherein the stability of the modified and unmodified Env proteins is determined by determining yield of the proteins in a recombinant protein expression system.

50. The method of claim 37, wherein an increased stability of the modified Env protein as compared to the unmodified Env protein results from a lower probability that the modified Env protein will assume a postfusion conformation as compared to the unmodified protein.

51. The method of claim 37, wherein an increased stability of the modified Env protein as compared to the unmodified Env protein results from a decreased metastability of the modified protein as compared to the unmodified protein.

52. A method for modifying an envelope protein (Env) from a retrovirus, comprising:
identifying amino acid positions within flexible loops of an unmodified Env protein;
substituting one or more of the identified amino acid positions in the unmodified Env protein with a proline to produce a modified Env; and
determining yield of the modified Env in an expression system.

53. The method of claim 52, wherein the flexible loops of the unmodified Env protein comprise loops within a gp41 subunit which are unstructured in a prefusion conformation of the unmodified protein and rearrange into alpha helices in a postfusion conformation of the unmodified protein.

54. The method of claim 52, further comprising selecting a modified Env from the method that produces a greater yield than the unmodified Env under similar experimental conditions.

55. A method for modifying an envelope protein (Env) of HIV-1, HIV-2 or SIV, comprising:
selecting an Env protein that has a threonine at position 568, a leucine at position 569, or a threonine at position 568 and a leucine at position 569; and
substituting the threonine at position 568, the leucine at position 569, or the threonine at position 568 and the leucine at position 569 with proline.

56. A method for modifying an envelope protein (Env) of HIV, comprising:
identifying amino acids at position 568, 569, or 568 and 569 in the Env; and
substituting one or more of the amino acid(s) with proline or glycine.

57. A vaccine composition comprising the modified type I fusion protein of any one of claim 1-34 or the modified HIV envelope protein of claim 35.

58. A nucleic acid encoding the modified type I fusion proteins of any one of claims 1-34 or the modified HIV envelope protein of claim 35.

59. A vector comprising the nucleic acid of claim 58.

60. A cell expressing the vector of claim 59.

61. A vaccine composition comprising the nucleic acid of claim 58, the vector of claim 59, the cell of claim 60, and combinations thereof.


SEQUENCE LISTING (18 sequences)

SEQ ID NO: 1 - protein, 648 aa, synthetic construct
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKEKKEYALFYKPDIVPLSETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKPPVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 2 - protein, 660 aa, synthetic construct
MPMGSLQPLATLYLLGMLVASVLASDEWVTVYYGVPVWREVNTVLFCASDAKAHSTEAHNIWATHACVPTDPNPQEVVLHNVTEDFNMWNNQMVEQMQEDISSLWDQSLKPCVKLTPLCVTMKCSNVTRQNSTSNSNNNPTLKNNTSGKNETGVLQMKNCTFNTTTELRDKKKKVYSLFYVDDLQSLGSGTGDTYTMINCNTTACTQACPKVSFEPIPIHYCAPAGFAILKCNDVNFSGKGKCRNVSTVHCTHGIKPVVTTQLIINGSLATENITVRVNNASKNTHDWIVQLSTAVNLTCKRVGNNTRGKVQIGPGMTFYNMDHIFGDTRKAFCELNGTTWNETLQKVRESLIKEIKANANGTYNITFEPSSGGDPEIANHMFNCGGEFFYCDTRKMFNESEPFHENMTIPCRIRQIVNSWMRVGRCIYAPPIPGHITCNSLITGLILTRDHVNNTNNTFRPIGGDMKNIWRSELYKYKVVRIEPLSVAPTKCKRHTVGERRRRRRAAFGLGALFLGFLGAAGSTMGAAAVTLTVQARQLLSGIVQQQNNLLRAPEAQQHLLQPPVWGVKQLQARLLAVERYLQDQQILGLWGCSGKSICCTTVPWNKTWSGKSMSDIWNNLTWQQWDKLITNYTGTIFGLLEEAQSQQEKNEKDLLELD

SEQ ID NO: 3 - protein, 666 aa, synthetic construct
MPMGSLQPLATLYLLGMLVASVLAAAKKWVTVYYGVPVWKEATTTLFCASDAKAYDTEVHNVWATHACVPTDPNPQEIVLGNVTENFNMWKNNMVEQMHEDIISLWDQSLKPCVKLTPLCVTLNCNNVNTNNTNNSTNATISDWEKMETGEMKNCSFNVTTSIRDKIKKEYALFYKLDVVPLENKNNINNTNITNYRLINCNTSVITQACPKVSFEPIPIHYCAPAGFAILKCNSKTFNGSGPCTNVSTVQCTHGIRPVVSTQLLLNGSLAEEEIVIRSENITDNAKTIIVQLNEAVEINCTRPNNNTRKSIHIGPGRAFYATGDIIGNIRQAHCNISKARWNETLGQIVAKLEEQFPNKTIIFNHSSGGDPEIVTHSFNCGGEFFYCNTTPLFNSTWNNTRTDDYPTGGEQNITLQCRIKQIINMWQGVGKAMYAPPIRGQIRCSSNITGLLLTRDGGRDQNGTETFRPGGGNMRDNWRSELYKYKVVKIEPLGIAPTACKRRVVQRRRRRRAVGLGAFILGFLGAAGSTMGAASMALTVQARLLLSGIVQQQNNLLRAPEAQQHMLQPPVWGIKQLQARVLAVERYLRDQQLLGIWGCSGKIICCTNVPWNDSWSNKTINEIWDNMTWMQWEKEIDNYTQHIYTLLEVSQIQQEKNEQELLELD

SEQ ID NO: 4 - protein, 652 aa, synthetic construct
MPMGSLQPLATLYLLGMLVASCLGVEKLWVTVYYGVPVWKEACTTLFCASDAKAYDTKVRNVWATHCCVPTDPNPQEVVLENVTEHFNMWKNNMVEQMQEDIISLWDQSLKPCVKLTPLCVTLNCKDVNATNTTNDSEGTMERGEIKNCSFNITTSIRDKVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRWFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEIGKAMYAPPIRGQIRCSSNITGLLLTRDGGINENGTEIFRPGGGDMRDNWRSELYKYKVVKIEPLGVAPTKCKRRVVGRRRRRRAVGIGAVFLGFLGAAGSTMGAASMTLTVQARQLLSGIVQQQNNCLRAPECQQRMLQPPVWGIKQLQARVLAVERYLGDQQLLGIWGCSGKLICCTAVPWNASWSNKSLDRIWNNMTWMEWEREIDNYTSEIYTLIEESQNQQEKNEQELLELD

SEQ ID NO: 5 - protein, 665 aa, synthetic construct
MDAMKRGLCCVLLLCGAVFVSPSQEIHARFRRGARAEKLWVTVYYGVPVWREADTTLFCASDAKGYDTEAHNVWATHACVPTDPRPQEMYLENVTENFNMWKNSMVEQMHTDIISLWDESLKPCVKLTPLCVTLDCQAFNSSSHTNSSIAMQEMKNCSFNVTTELRDKKKKEYSFFYKTDIEQINKNGRQYRLINCNTSACTQACPKVSFEPIPIHFCAPAGFAILKCNEKHFNGKGPCKNVSTVQCTHGIKPVVSTQLLLNGSLAEEEVVIRVENTIDNAKTIIVQLAKPVKINCTRPNNNTRKSIRIGPGQTFYATGDIIGNIRKAYCNVSKREWNNTLQQVAAQLSKSFNNTKIVFEKHSGGDLEVITHSFVCGGEFFYCNTSGLFNSTWTNSTWTNSTTGSNGTESNDTITLQCEIKQFINMWQRVGRCMYAPPIPGVIRCESDITGLLLTRDGPNSTQNETFRPGGGDMRDNWRSELYKYKVVQIEPLGVAPTHCKRRVVERRRRRRAVGLGAVFFGFLGAAGSTMGAASITLTVQARQLLSGIVQQQSNLLKAPEAQQQLLRPPVWGIKQLQARVLALERYLKDQQLLGIWGCSGKLICCTTVPWNSSWSNKNYTDIWDNMTWLQWDREISNYTDEIYRLIEQSQNQQEKNEQDLLALD

SEQ ID NO: 6 - protein, 653 aa, synthetic construct
MPMGSLQPLATLYLLGMLVASVLAAENLWVTVYYGVPVWKEAKTTLFCASDAKAYEKEVHNIWATHACVPTDPNPQEMVLKNVTENFNMWKNDMVDQMHEDVISLWDQSLKPCVKLTPLCVTLNCTNATANATASNSSIIEGMKNCSFNITTELRDKREKKNALFYKLDIVQLDGNSSQYRLINCNTSVITQVCPKVSFDPIPIHYCAPAGYAILKCNNKTFTGTGPCNNVSTVQCTHGIKPVLSTQLLLNGSLAEGEIIIRSENITNNGKTIIVQLNESVKIECTRPNNKTRTSIRIGPGQAFYATGQVIGNIREAYCNISESKWNETLQRVSKKLKEYFPHKNITFQPSSGGDLEITTHSFNCGGEFFYCNTSSLFNRTYMANSTDMANSTETNSTRIITIHCRIKQIINMWQEVGRAMYAPPIAGNITCISNITGLLLTRDGGKNNTDTETFRPGGGNMKDNWRSELYKYKVVEVKPLGVAPTNCRRRVVERRRRRRAVGMGAVFLGFLGAAGSTMGAASITLTVQARQLLSGIVQQQSNLLKAPEAQQHMLKPPVWGIKQLQARVLALERYLKDQQLLGMWGCSGKLICCTNVYWNSSWSNKTYGDIWDNMTWMQWEREISNYTEIIYELLEESQNQQEKNEQDLLALD

SEQ ID NO: 7 - protein, 708 aa, synthetic construct
MAISGVPVLGFFIIAVLMSAQESWAGLWVTVYYGVPVWREAKTTLFCASDAKSYEKEVANIWATHACVPTDPNPQELVLKNVTENFNMWKNDMVDQMHEDIISLWDQSLKPCVKLTPLCVTLHCSTYNNTHNISKEIKNCSFNATTELRDKRRKEYALFYRLDIVPLNKNGRQYRLINCNTSVCTQVCPKLTFDPIPIHYCAPAGYAILKCNNKTFNGTGPCNNVSTVQCTHGIKPVLSTQLLLNGSLAEEEIIIRSENLTDNVKTIIVHLNESVEINCTRPNNNTRKSIRIGPGQTFYATGDIIGDIRQAHCNISEIKWNKTLQRVSEKLREHFNKTIIFNQSSGGDLEITTHSFNCGGEFFYCNTSDLFFNKTFNETYSTGSNSTNSNITLPCRIKQIINMWQEVGRCMYASPIAGEITCKSNITGLLLTRDGGGNNSTEETFRPGGGNMRDNWRSELYKYKVVEVKPLGIAPTECRRRVVQGGGGSGGGGSAVVGLGAVFLGFLGTAGSTMGAASITLTVQARQLLSGIVQQQSNLLRAPEAQQHMLQPPVWGIKQLQARVLTIERYAKDQQLLGMWGCSGKLICCTNVYWNSSWSNKTYNEIWDNMTWMQWDREIDNYTDTIYKLLEVSQKQQESNEKDLLALDSWNNLWNWFDISKWLWYIKIFIMIVGGLIGLRIIFAVLSLVNRVRQGIRPVFSSPPSYFQ

SEQ ID NO: 8 - protein, 708 aa, synthetic construct
MAISGVPVLGFFIIAVLMSAQESWAGLWVTVYYGVPVWREAKTTLFCASDAKSYEKEVANIWATHACVPTDPNPQELVLKNVTENFNMWKNDMVDQMHEDIISLWDQSLKPCVKLTPLCVTLHCSTYNNTHNISKEIKNCSFNATTELRDKRRKEYALFYRLDIVPLNKNGRQYRLINCNTSVCTQVCPKLTFDPIPIHYCAPAGYAILKCNNKTFNGTGPCNNVSTVQCTHGIKPVLSTQLLLNGSLAEEEIIIRSENLTDNVKTIIVHLNESVEINCTRPNNMTRKSIRIGPGQTFYALGDIIGDIRQPHCNISEIKWNKTLQRVSEKLREHFNKTIIFNQSSGGDLEITTHSFNCGGEFFYCNTSDLFFNKTFNETYSTGSNSTNSNITLPCRIKQIINMWQEVGRCMYAPPIAGNITCKSNITGLLLTRDGGGNNSTEETFRPGGGNMRDNWRSELYKYKVVEVKPLGIAPTECRRRVVQGGGGSGGGGSAVVGLGAVFLGFLGTAGSTMGAASNTLTVQARQLLSGIVQQQSNLLRAPEAQQHMLQPPVWGFKQLQARVLTIERYAEVQQLLGMWGCSGKLICCTNVPWNSSWSNKTYNEIWDNMTWMQWDREIGNYTDTIYKLLEVSQFQQEINEKDLLALDSWNNLWNWFDISKWLWYIKIFIMIVGGLIGLRIIFAVLSLVNRVRQGIRPVFSSPPSYFQ

SEQ ID NO: 9 - protein, 708 aa, synthetic construct
MAISGVPVLGFFIIAVLMSAQESWAGLWVTVYYGVPVWREAKTTLFCASDAKSYEKEVHNVWATHACVPTDPNPQELVLKNVTENFNMWKNDMVDQMHEDIISLWDQSLKPCVKLTPLCVTLHCSTYNNTHNISKEIKNCSFNATTELRDKRRKEYALFYRLDIVPLNKNGRQYRLINCNTSVCTQICPKVTFDPIPIHYCAPAGYAILKCNNKTFNGTGPCNNVSTVQCTHGIKPVVSTQLLLNGSLAEEEIIIRSENLTDNVKTIIVHLNESVEINCTRPNNMTRKSIRIGPGQTFYALGDIIGDIRQPHCNISEIKWNKTLQRVSEKLREHFNKTIIFNQSSGGDLEITTHSFNCGGEFFYCNTSDLFFNKTFNETYSTGSNSTNSNITLPCRIKQIINMWQEVGRCMYAPPIAGNITCKSNITGLLLTRDGGGNNSTEETFRPGGGNMRDNWRSELYKYKVVEVKPLGIAPTECRRRVVQGGGGSGGGGSAVVGLGAVFLGFLGTAGSTMGAASNTLTVQARQLLSGIVQQQSNLLRAPEAQQHMLQPPVWGFKQLQARVLAIERYLEVQQLLGMWGCSGKLICCTNVPWNSSWSNKTYNEIWDNMTWMQWDREIGNYTDTIYKLLEVSQFQQEINEKDLLALDSWNNLWNWFDISKWLWYIKIFIMIVGGLIGLRIIFAVLSLVNRVRQGIRPVFSSPPSYFQ

SEQ ID NO: 10 - DNA, 2547 bp, synthetic construct
  source 1..2547: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatggccatctccggcgtgcccgtgctgggcttcttcatcatcgccgtgctgatgtccgcccaggagtcctgggccggcctgtgggtgaccgtgtactacggcgtgcccgtgtggcgcgaggccaagaccaccctgttctgcgcctccgacgccaagtcctacgagaaggaggtggccaacatctgggccacccacgcctgcgtgcccaccgaccccaacccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgcactgctccacctacaacaacacccacaacatctccaaggagatcaagaactgctccttcaacgccaccaccgagctgcgcgacaagcgccgcaaggagtacgccctgttctaccgcctggacatcgtgcccctgaacaagaacggccgccagtaccgcctgatcaactgcaacacctccgtgtgcacccaggtgtgccccaagctgaccttcgaccccatccccatccactactgcgcccccgccggctacgccatcctgaagtgcaacaacaagaccttcaacggcaccggcccctgcaacaacgtgtccaccgtgcagtgcacccacggcatcaagcccgtgctgtccacccagctgctgctgaacggctccctggccgaggaggagatcatcatccgctccgagaacctgaccgacaacgtgaagaccatcatcgtgcacctgaacgagtccgtggagatcaactgcacccgccccaacaacaacacccgcaagtccatccgcatcggccccggccagaccttctacgccaccggcgacatcatcggcgacatccgccaggcccactgcaacatctccgagatcaagtggaacaagaccctgcagcgcgtgtccgagaagctgcgcgagcacttcaacaagaccatcatcttcaaccagtcctccggcggcgacctggagatcaccacccactccttcaactgcggcggcgagttcttctactgcaacacctccgacctgttcttcaacaagaccttcaacgagacctactccaccggctccaactccaccaactccaacatcaccctgccctgccgcatcaagcagatcatcaacatgtggcaggaggtgggccgctgcatgtacgcctcccccatcgccggcgagatcacctgcaagtccaacatcaccggcctgctgctgacccgcgacggcggcggcaacaactccaccgaggagaccttccgccccggcggcggcaacatgcgcgacaactggcgctccgagctgtacaagtacaaggtggtggaggtgaagcccctgggcatcgcccccaccgagtgccgccgccgcgtggtgcagggcggcggcggctccggcggcggcggctccgccgtggtgggcctgggcgccgtgttcctgggcttcctgggcaccgccggctccaccatgggcgccgcctccatcaccctgaccgtgcaggcccgccagctgctgtccggcatcgtgcagcagcagtccaacctgctgcgcgcccccgaggcccagcagcacatgctgcagccccccgtgtggggcatcaagcagctgcaggcccgcgtgctgaccatcgagcgctacgccaaggaccagcagctgctgggcatgtggggctgctccggcaagctgatctgctgcaccaacgtgtactggaactcctcctggtccaacaagacctacaacgagatctgggacaacatgacctggatgcagtgggaccgcgagatcgacaactacaccgacaccatctacaagctgctggaggtgtcccagaagcagcaggagtccaacgagaaggacctgctggccctggactcctggaacaacctgtggaactggttcgacatctccaagtggctgtggtacatcaagatcttcatcatgatcgtgggcggcctgatcggcctgcgcatcatcttcgccgtgctgtccctggtgaaccgcgtgcgccagggcatccgccccgtgttctcctcccccccctcctacttccagtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 2547 bp, synthetic construct
  source 1..2547: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatggccatctccggcgtgcccgtgctgggcttcttcatcatcgccgtgctgatgtccgcccaggagtcctgggccggcctgtgggtgaccgtgtactacggcgtgcccgtgtggcgcgaggccaagaccaccctgttctgcgcctccgacgccaagtcctacgagaaggaggtggccaacatctgggccacccacgcctgcgtgcccaccgaccccaacccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgcactgctccacctacaacaacacccacaacatctccaaggagatcaagaactgctccttcaacgccaccaccgagctgcgcgacaagcgccgcaaggagtacgccctgttctaccgcctggacatcgtgcccctgaacaagaacggccgccagtaccgcctgatcaactgcaacacctccgtgtgcacccaggtgtgccccaagctgaccttcgaccccatccccatccactactgcgcccccgccggctacgccatcctgaagtgcaacaacaagaccttcaacggcaccggcccctgcaacaacgtgtccaccgtgcagtgcacccacggcatcaagcccgtgctgtccacccagctgctgctgaacggctccctggccgaggaggagatcatcatccgctccgagaacctgaccgacaacgtgaagaccatcatcgtgcacctgaacgagtccgtggagatcaactgcacccgccccaacaacatgacccgcaagtccatccgcatcggccccggccagaccttctacgccctgggcgacatcatcggcgacatccgccagccccactgcaacatctccgagatcaagtggaacaagaccctgcagcgcgtgtccgagaagctgcgcgagcacttcaacaagaccatcatcttcaaccagtcctccggcggcgacctggagatcaccacccactccttcaactgcggcggcgagttcttctactgcaacacctccgacctgttcttcaacaagaccttcaacgagacctactccaccggctccaactccaccaactccaacatcaccctgccctgccgcatcaagcagatcatcaacatgtggcaggaggtgggccgctgcatgtacgccccccccatcgccggcaacatcacctgcaagtccaacatcaccggcctgctgctgacccgcgacggcggcggcaacaactccaccgaggagaccttccgccccggcggcggcaacatgcgcgacaactggcgctccgagctgtacaagtacaaggtggtggaggtgaagcccctgggcatcgcccccaccgagtgccgccgccgcgtggtgcagggcggcggcggctccggcggcggcggctccgccgtggtgggcctgggcgccgtgttcctgggcttcctgggcaccgccggctccaccatgggcgccgcctccaacaccctgaccgtgcaggcccgccagctgctgtccggcatcgtgcagcagcagtccaacctgctgcgcgcccccgaggcccagcagcacatgctgcagccccccgtgtggggcttcaagcagctgcaggcccgcgtgctgaccatcgagcggtacgccgaggtgcagcagctgctgggcatgtggggctgctccggcaagctgatctgctgcaccaacgtgccctggaactcctcctggtccaacaagacctacaacgagatctgggacaacatgacctggatgcagtgggaccgcgagatcggcaactacaccgacaccatctacaagctgctggaggtgtcccagttccagcaggagatcaacgagaaggacctgctggccctggactcctggaacaacctgtggaactggttcgacatctccaagtggctgtggtacatcaagatcttcatcatgatcgtgggcggcctgatcggcctgcgcatcatcttcgccgtgctgtccctggtgaaccgcgtgcgccagggcatccgccccgtgttctcctcccccccctcctacttccagtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 2547 bp, synthetic construct
  source 1..2547: mol_type = other DNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgctgccaccatggccatctccggcgtgcccgtgctgggcttcttcatcatcgccgtgctgatgtccgcccaggagtcctgggccggcctgtgggtgaccgtgtactacggcgtgcccgtgtggcgcgaggccaagaccaccctgttctgcgcctccgacgccaagtcctacgagaaggaggtgcacaacgtgtgggccacccacgcctgcgtgcccaccgaccccaacccccaggagctggtgctgaagaacgtgaccgagaacttcaacatgtggaagaacgacatggtggaccagatgcacgaggacatcatctccctgtgggaccagtccctgaagccctgcgtgaagctgacccccctgtgcgtgaccctgcactgctccacctacaacaacacccacaacatctccaaggagatcaagaactgctccttcaacgccaccaccgagctgcgcgacaagcgccgcaaggagtacgccctgttctaccgcctggacatcgtgcccctgaacaagaacggccgccagtaccgcctgatcaactgcaacacctccgtgtgcacccagatctgccccaaggtgaccttcgaccccatccccatccactactgcgcccccgccggctacgccatcctgaagtgcaacaacaagaccttcaacggcaccggcccctgcaacaacgtgtccaccgtgcagtgcacccacggcatcaagcccgtggtgtccacccagctgctgctgaacggctccctggccgaggaggagatcatcatccgctccgagaacctgaccgacaacgtgaagaccatcatcgtgcacctgaacgagtccgtggagatcaactgcacccgccccaacaacatgacccgcaagtccatccgcatcggccccggccagaccttctacgccctgggcgacatcatcggcgacatccgccagccccactgcaacatctccgagatcaagtggaacaagaccctgcagcgcgtgtccgagaagctgcgcgagcacttcaacaagaccatcatcttcaaccagtcctccggcggcgacctggagatcaccacccactccttcaactgcggcggcgagttcttctactgcaacacctccgacctgttcttcaacaagaccttcaacgagacctactccaccggctccaactccaccaactccaacatcaccctgccctgccgcatcaagcagatcatcaacatgtggcaggaggtgggccgctgcatgtacgccccccccatcgccggcaacatcacctgcaagtccaacatcaccggcctgctgctgacccgcgacggcggcggcaacaactccaccgaggagaccttccgccccggcggcggcaacatgcgcgacaactggcgctccgagctgtacaagtacaaggtggtggaggtgaagcccctgggcatcgcccccaccgagtgccgccgccgcgtggtgcagggcggcggcggctccggcggcggcggctccgccgtggtgggcctgggcgccgtgttcctgggcttcctgggcaccgccggctccaccatgggcgccgcctccaacaccctgaccgtgcaggcccgccagctgctgtccggcatcgtgcagcagcagtccaacctgctgcgcgcccccgaggcccagcagcacatgctgcagccccccgtgtggggcttcaagcagctgcaggcccgcgtgctggccatcgagcgctacctggaggtgcagcagctgctgggcatgtggggctgctccggcaagctgatctgctgcaccaacgtgccctggaactcctcctggtccaacaagacctacaacgagatctgggacaacatgacctggatgcagtgggaccgcgagatcggcaactacaccgacaccatctacaagctgctggaggtgtcccagttccagcaggagatcaacgagaaggacctgctggccctggactcctggaacaacctgtggaactggttcgacatctccaagtggctgtggtacatcaagatcttcatcatgatcgtgggcggcctgatcggcctgcgcatcatcttcgccgtgctgtccctggtgaaccgcgtgcgccagggcatccgccccgtgttctcctcccccccctcctacttccagtaataaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - protein, 25 aa, synthetic construct
MAISGVPVLGFFIIAVLMSAQESWA

SEQ ID NO: 14 - protein, 12 aa, synthetic construct
RPVFSSPPSYFQ

SEQ ID NO: 15 - RNA, 145 nt, synthetic construct
  source 1..145: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgct

SEQ ID NO: 16 - RNA, 165 nt, synthetic construct
  source 1..165: mol_type = other RNA
actagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattct

SEQ ID NO: 17 - RNA, 101 nt, synthetic construct
  source 1..101: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 8 aa, synthetic construct
HHHHHHHH